(19) United States
(12) Patent Application Publication
(10) Pub. No.: US 2024/0207392 A1
(43) Pub. Date: Jun. 27, 2024

(54) EPSTEIN-BARR VIRUS MRNA VACCINES

(71) Applicant: ModernaTX, Inc., Cambridge, MA (US)

(72) Inventors: Sumana Chandramouli, Cambridge, MA (US); Brooke Bollman, Brookline, MA (US); Yen-Ting Lai, Cambridge, MA (US); Guillaume Stewart-Jones, Cambridge, MA (US); Andrea Carfi, Cambridge, MA (US)

(73) Assignee: ModernaTX, Inc., Cambridge, MA (US)

(21) Appl. No.: 18/286,705

(22) PCT Filed: Apr. 13, 2022
(86) PCT No.: PCT/US2022/024530

§ 371 Date: Oct. 12, 2023

Related U.S. Application Data
(60) Provisional application No. 63/174,287, filed on Apr. 13, 2021.

(51) Int. Cl. A61K 39/245; A61K 9/51; A61K 39/00; A61P 31/20; A61P 37/04

(52) U.S. Cl. CPC A61K 39/245; A61K 9/5123; A61P 31/20; A61P 37/04; A61K 2039/53; A61K 2039/6018

(57) ABSTRACT

The disclosure relates to Epstein-Barr virus ribonucleic acid vaccines as well as methods of using the vaccines and compositions comprising the vaccines.

### RELATED APPLICATION

This application claims the benefit under 35 U.S.C. § 119(c) of U.S. provisional application No. 63/174,287, filed Apr. 13, 2021, which is incorporated by reference herein in its entirety.

### BACKGROUND

Epstein-Barr virus (EBV), also referred to as human herpesvirus 4, is a common herpesvirus that is spread through bodily fluids, most commonly saliva, and contracted primarily by young children and adolescents (approximately 50% and approximately 89% seropositivity, respectively). It is a major cause of infectious mononucleosis in the U.S., accounting for over 90% of the approximately 1-2 million cases annually. Infectious mononucleosis can debilitate patients for weeks to months and, in some cases, can lead to hospitalization and splenic rupture. EBV infection is associated with the development and progression of certain lymphoproliferative disorders, cancers, and an increased risk of autoimmune diseases including multiple sclerosis, an autoimmune disease of the central nervous system. There is no approved vaccine for EBV.

### SUMMARY

A messenger ribonucleic acid (mRNA)-based vaccine platform has been developed based on the principle and observations that target viral proteins (antigens) can be produced in vivo by delivery and cellular uptake of the corresponding synthetic viral mRNA from delivery of a vaccine comprising the mRNA formulated in a lipid nanoparticle. The mRNA then undergoes intracellular ribosomal translation to endogenously express the viral proteins encoded by the vaccine comprising synthetic viral mRNA. These mRNA-based vaccines do not enter the cellular nucleus or interact with the human genome, are nonreplicating, and are expressed transiently. mRNA vaccines offer a mechanism to stimulate the endogenous production in human cells of multiple structurally intact, properly folded and glycosylated viral glycoproteins, for example, in a manner that mimics wild-type viral infection and is able to induce highly targeted immune responses against infectious pathogens such as EBV.

Some aspects of the present disclosure provide a vaccine, comprising a lipid nanoparticle that comprises: (a) a messenger ribonucleic acid (mRNA) comprising an open reading frame encoding an Epstein-Barr virus (EBV) glycoprotein 220 (gp220); (b) an mRNA comprising an open reading frame encoding glycoprotein 42 (gp42); (c) an mRNA comprising an open reading frame encoding glycoprotein L (gL); and (d) an mRNA comprising an open reading frame encoding glycoprotein H (gH).

In some embodiments, the gp42 is a soluble form of gp42.

In some embodiments, the mass ratio of (a):(b):(c):(d) is 4:1:1:1.5. In other embodiments, the mass ratio of (a):(b):(c):(d) is 4:1:1:1.

In some embodiments, the vaccine of claim 1 or 2, wherein mRNA of (a) is at 5×, 4.5×, 4×, 3.5×, 3×, 2.5×, 2×, or 1.5× the mRNA of (b), (c), and/or (d). In some embodiments, the mRNA of (a) is at equal mass to the mRNA of (b), (c), and/or (d).

In some embodiments, the mRNA of (b) is at 5×, 4.5×, 4×, 3.5×, 3×, 2.5×, 2×, or 1.5× the mRNA of (a), (c), and/or (d). In some embodiments, the mRNA of (b) is at equal mass to the mRNA of a), (c), and/or (d).

In some embodiments, the mRNA of (c) is at 5×, 4.5×, 4×, 3.5×, 3×, 2.5×, 2×, or 1.5× the mRNA of (a), (b), and/or (d). In some embodiments, the mRNA of (c) is at equal mass to the mRNA of (a), (b), and/or (d).

In some embodiments, the mRNA of (d) is at 5×, 4.5×, 4×, 3.5×, 3×, 2.5×, 2×, or 1.5× the mRNA of (a), (b), and/or (c). In some embodiments, the mRNA of (d) is at equal mass to the mRNA of (a), (b), and/or (c).

In some embodiments, the vaccine comprises 26.7 μg mRNA encoding EBV gp220, 6.7 μg mRNA encoding EBV gp42, 6.7 μg mRNA encoding EBV gL, and 10 μg mRNA encoding EBV gH. In other embodiments, the vaccine comprises 53.3 μg mRNA encoding EBV gp220, 13.3 μg mRNA encoding EBV gp42, 13.3 μg mRNA encoding EBV gL, and 20 μg mRNA encoding EBV gH. In still other embodiments, the vaccine comprises 106.7 μg mRNA encoding EBV gp220, 26.7 μg mRNA encoding EBV gp42, 26.7 μg mRNA encoding EBV gL, and 40 μg mRNA encoding EBV gH

In some embodiments, the lipid nanoparticle further comprises (e) an mRNA comprising an open reading frame encoding glycoprotein B. In some embodiments, the mass ratio of (a):(b):(c):(d):(c) is 4:1:1:1.5:1.5.

In some embodiments, the gp220 comprises an amino acid sequence having at least 85%, at least 90%, at least 95%, or at least 98% identity to the amino acid sequence of SEQ ID NO: 4. In some embodiments, the gp220 comprises the amino acid sequence of SEQ ID NO: 4.

In some embodiments, the gL comprises an amino acid sequence having at least 85%, at least 90%, at least 95%, or at least 98% identity to the amino acid sequence of SEQ ID NO: 8. In some embodiments, the gL comprises the amino acid sequence of SEQ ID NO: 8.

In some embodiments, the gH comprises an amino acid sequence having at least 85%, at least 90%, at least 95%, or at least 98% identity to the amino acid sequence of SEQ ID NO: 6. In some embodiments, the gH comprises the amino acid sequence of SEQ ID NO: 6.

In some embodiments, the gp42 comprises an amino acid sequence having at least 85%, at least 90%, at least 95%, or at least 98% identity to the amino acid sequence of SEQ ID NO: 14. In some embodiments, the gp42 comprises the amino acid sequence of SEQ ID NO: 14.

In some embodiments, the soluble gp42 comprises an amino acid sequence having at least 85%, at least 90%, at least 95%, or at least 98% identity to the amino acid sequence of SEQ ID NO: 10. In some embodiments, the soluble gp42 comprises the amino acid sequence of SEQ ID NO: 10.

In some embodiments, the gB comprises an amino acid sequence having at least 85%, at least 90%, at least 95%, or at least 98% identity to the amino acid sequence of SEQ ID NO: 12. In some embodiments, the gB comprises the amino acid sequence of SEQ ID NO: 12.

In some embodiments, the mRNA of any one or more of (a)-(e) comprises a 5′ 7mG(5′)ppp(5′)NlmpNp cap and a 3′ polyA tail.

In some embodiments, the mRNA of any one or more of (a)-(e) comprises a 1-methylpseudourine chemical modification.

In some embodiments, the lipid nanoparticle comprises 45-55 mol % ionizable amino lipid, 15-20 mol % neutral lipid, 35-45 mol % cholesterol, and 0.5-5 mol % PEG-modified lipid.

In some embodiments, the ionizable amino lipid is Compound I:

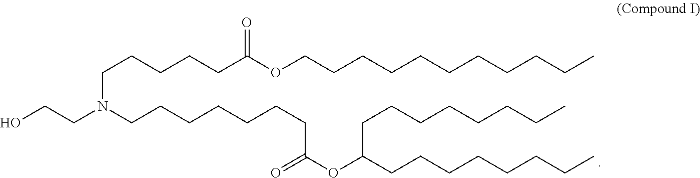 [SMILES: CCCCCCCCCCCOC(=O)CCCCCN(CCO)CCCCCCCC(=O)OC(CCCCCCCC)CCCCCCCC]

In some embodiments, the lipid nanoparticle comprises 50 mol % ionizable amino lipid. In other embodiments, the lipid nanoparticle comprises 49 mol % ionizable amino lipid. In yet other embodiments, the lipid nanoparticle comprises 48 mol % ionizable amino lipid.

Other aspects of the present disclosure provide a method comprising administering to a subject the vaccine of any one of the preceding claims in an amount effective to induce an immune response to EBV.

In some embodiments, the vaccine is administered as a single dose. In other embodiments, the vaccine is administered as an initial dose and as at least one booster dose.

### BRIEF DESCRIPTION OF THE DRAWINGS

FIG. 1 is a schematic depicting intracellular delivery of the EBV mRNA vaccine and expression of the encoded EBV glycoproteins at the cell surface.

FIGS. 2A-2B are graphs of data showing that the addition of mRNA encoding soluble EBV gp42 to a vaccine comprising mRNA encoding EBV gH, mRNA encoding EBV gL, and mRNA encoding gp220 (1) improves neutralizing antibody titers produced against B cell infection, similar to mRNA encoding wild-type (membrane-bound) gp42 (FIG. 2A) and (2) does not dampen neutralizing antibody titers produced against epithelial cell to the extent of mRNA encoding wild-type gp42 (FIG. 2B). The data also shows that the addition of mRNA encoding EBV gB to a vaccine comprising either mRNA encoding soluble gp42 or mRNA encoding wild-type gp42 leads to a significant drop in B cell neutralizing antibody titers (FIG. 2A).

FIGS. 3A-3B include graphs showing neutralizing antibodies against B cell infection (FIG. 3A) and against epithelial cell infection (FIG. 3B) observed in preclinical studies.

FIGS. 4A-4B include graphs showing antibodies against B cell infection (FIG. 4A) and against epithelial cell infection (FIG. 4B) observed in preclinical studies using Balb/c mice and two different doses of mRNA vaccine: 10 μg (5.33 μg mRNA encoding EBV gp220, 2 μg mRNA encoding EBV gH, 1.33 μg mRNA encoding EBV gL, 1.33 μg mRNA encoding EBV sgp42) and 2.5 μg (1.33 μg mRNA encoding EBV gp220, 0.5 μg mRNA encoding EBV gH, 0.33 μg mRNA encoding EBV gL, 0.33 μg mRNA encoding EBV sgp42). The mRNA vaccine was immunogenic at both doses, raising B cell and epithelial cell neutralizing antibodies (gHgL, gp220 and gp42).

FIGS. 5A-5C include graphs showing IgG titers in an ELISA assay using samples from Balb/c mice administered two different doses of mRNA vaccine: 10 μg (5.33 μg mRNA encoding EBV gp220, 2 μg mRNA encoding EBV gH, 1.33 μg mRNA encoding EBV gL, 1.33 μg mRNA encoding EBV sgp42) and 2.5 μg (1.33 μg mRNA encoding EBV gp220, 0.5 μg mRNA encoding EBV gH, 0.33 μg mRNA encoding EBV gL, 0.33 μg mRNA encoding EBV gp42). FIG. 5A: EBV gHgL; FIG. 5B: EBV gp220; FIG. 5C: EBV gp42. The mRNA vaccine was immunogenic at both doses, raising binding antibodies to all four antigens (gHgL, gp220 and sgp42).

FIGS. 6A-6C include graphs showing antigen-binding antibody titers from a 2-dose 35-day immunogenicity study in rats. The animals were administered the first dose on Day 1 and the second dose on Day 22, then antibody titers were assessed on Day 35. Three different doses were assessed: 30 μg total mRNA, 60 μg total mRNA, and 80 μg total mRNA (mRNA encoding EBV gp220, mRNA encoding EBVgH, mRNA encoding EBV gL, mRNA encoding EBV sgp42). N=10 (5 males and 5 females). FIG. 6A: EBV gHgL; FIG. 6B: EBV sgp42; FIG. 6C: EBV gp220. All doses evaluated elicited strong serum IgG responses against the vaccine antigens.

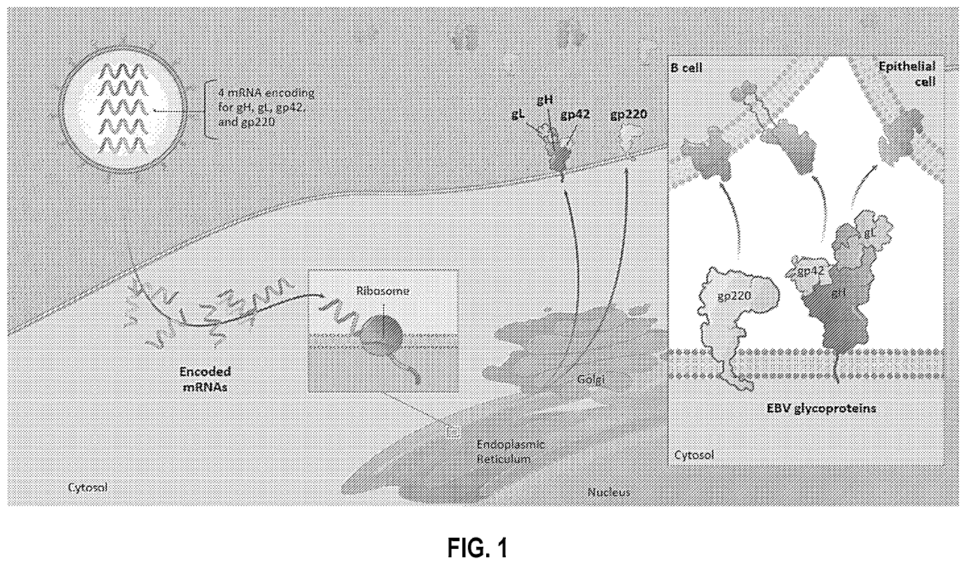

### DETAILED DESCRIPTION

EBV is a member of the herpesvirus family that includes CMV, is spread through bodily fluids (e.g., saliva) and contracted primarily by young children and adolescents. The EBV lifecycle has lytic and latent stages, similar to other herpesviruses such as CMV, as well as multiple surface (envelope) glycoproteins that mediate virus entry in different cell types. The mRNA vaccine against EBV as provide herein contains at least four mRNAs that encode viral proteins (gp220, gp42, gH and gL) in EBV. In some embodiments, these viral proteins are expressed in their native membrane-bound form for recognition by the immune system. In other embodiments, gp42 is expressed in a soluble form. In some embodiments, the vaccine further comprises an mRNA that encodes EBV glycoprotein B (gB).

The EBV mRNA vaccines described herein are superior to current vaccines in several ways. For example, the lipid nanoparticle (LNP) delivery system used herein increases the efficacy of mRNA vaccines in comparison to other formulations, including a protamine-based approach described in the literature. The use of this LNP delivery system enables the effective delivery of chemically-modified RNA vaccines or unmodified mRNA vaccines, without requiring additional adjuvant to produce a therapeutic result (e.g., production neutralizing antibody titer). In some embodiments, the EBV mRNA vaccines disclosed herein are superior to conventional vaccines by a factor of at least 10-fold, 20-fold, 40-fold, 50-fold, 100-fold, 500-fold, or 1,000-fold when administered intramuscularly (IM) or intradermally (ID). These results can be achieved even when significantly lower doses of the mRNA are administered in comparison with RNA doses used in other classes of lipid-based formulations.

Further, unlike self-replicating RNA vaccines, which rely on viral replication pathways to deliver enough RNA to a cell to produce an immunogenic response, the vaccines of the present disclosure do not require viral replication to produce enough protein to result in a strong immune response. Thus, the vaccines of the present disclosure do not include self-replicating RNA and do not include components necessary for viral replication.

It should be understood that the mRNA of the vaccines of the present disclosure are not naturally occurring. That is, the mRNA encoding the EBV antigen, as provided herein, does not occur in nature. It should also be understood that the vaccines described herein exclude viruses (i.e., the vaccines are not, nor do they contain, viruses).

### EBV Antigens

Antigens are proteins capable of inducing an immune response (e.g., causing an immune system to produce antibodies against the antigens). Herein, use of the term “antigen” encompasses immunogenic/antigenic proteins and immunogenic/antigenic fragments (e.g., an immunogenic/antigenic fragment that induces (or is capable of inducing) an immune response to human EBV). It should also be understood that the term “protein” encompasses full length proteins, truncated proteins, modified proteins, and peptides.

Exemplary nucleic acid and amino acids sequences of the EBV antigen of the vaccine provided herein are provided in Table 3.

In some embodiments, the gp220 comprises an amino acid sequence having at least 85% identity to the amino acid sequence of SEQ ID NO: 4. In some embodiments, the gp220 comprises an amino acid sequence having at least 90% identity to the amino acid sequence of SEQ ID NO: 4. In some embodiments, the gp220 comprises an amino acid sequence having at least 95% identity to the amino acid sequence of SEQ ID NO: 4. In some embodiments, the gp220 comprises an amino acid sequence having at least 98% identity to the amino acid sequence of SEQ ID NO: 4. In some embodiments, the gp220 comprises the amino acid sequence of SEQ ID NO: 4.

In some embodiments, the gL comprises an amino acid sequence having at least 85% identity to the amino acid sequence of SEQ ID NO: 8. In some embodiments, the gL comprises an amino acid sequence having at least 90% identity to the amino acid sequence of SEQ ID NO: 8. In some embodiments, the gL comprises an amino acid sequence having at least 95% identity to the amino acid sequence of SEQ ID NO: 8. In some embodiments, the gL comprises an amino acid sequence having at least at least 98% identity to the amino acid sequence of SEQ ID NO: 8.

In some embodiments, the gL comprises the amino acid sequence of SEQ ID NO: 8.

In some embodiments, the gH comprises an amino acid sequence having at least 85% identity to the amino acid sequence of SEQ ID NO: 6. In some embodiments, the gH comprises an amino acid sequence having at least 90% identity to the amino acid sequence of SEQ ID NO: 6. In some embodiments, the gH comprises an amino acid sequence having at least 95% identity to the amino acid sequence of SEQ ID NO: 6. In some embodiments, the gH comprises an amino acid sequence having at least 98% identity to the amino acid sequence of SEQ ID NO: 6. In some embodiments, the gH comprises the amino acid sequence of SEQ ID NO: 6.

In some embodiments, the gp42 comprises an amino acid sequence having at least 85% identity to the amino acid sequence of SEQ ID NO: 14. In some embodiments, the gp42 comprises an amino acid sequence having at least 90% identity to the amino acid sequence of SEQ ID NO: 14. In some embodiments, the gp42 comprises an amino acid sequence having at least 95% identity to the amino acid sequence of SEQ ID NO: 14. In some embodiments, the gp42 comprises an amino acid sequence having at least 98% identity to the amino acid sequence of SEQ ID NO: 14. In some embodiments, the gp42 comprises the amino acid sequence of SEQ ID NO: 14.

In some embodiments, the soluble gp42 comprises an amino acid sequence having at least 85% identity to the amino acid sequence of SEQ ID NO: 10. In some embodiments, the soluble gp42 comprises an amino acid sequence having at least 90% identity to the amino acid sequence of SEQ ID NO: 10. In some embodiments, the soluble gp42 comprises an amino acid sequence having at least 95% identity to the amino acid sequence of SEQ ID NO: 10. In some embodiments, the soluble gp42 comprises an amino acid sequence having at least 98% identity to the amino acid sequence of SEQ ID NO: 10. In some embodiments, the soluble gp42 comprises the amino acid sequence of SEQ ID NO: 10.

In some embodiments, the gB comprises an amino acid sequence having at least 85% identity to the amino acid sequence of SEQ ID NO: 12. In some embodiments, the gB comprises an amino acid sequence having at least 90% identity to the amino acid sequence of SEQ ID NO: 12. In some embodiments, the gB comprises an amino acid sequence having at least 95% identity to the amino acid sequence of SEQ ID NO: 12. In some embodiments, the gB comprises an amino acid sequence having at least 98% identity to the amino acid sequence of SEQ ID NO:

12. In some embodiments, the gB comprises the amino acid sequence of SEQ ID NO: 12.

It should be understood that the mRNA F protein described herein may or may not comprise a signal sequence.

### EBV Nucleic Acids

The vaccines of the present disclosure comprise a (at least one) mRNA having an open reading frame (ORF) encoding an EBV antigen. In some embodiments, the mRNA further comprises a 5′ UTR, 3′ UTR, a poly(A) tail and/or a 5′ cap analog.

It should also be understood that the mRNA vaccine of the present disclosure may include any 5′ untranslated region (UTR) and/or any 3′ UTR. Exemplary UTR sequences are provided in the Sequence Listing (e.g., SEQ ID NOs: 2-5); however, other UTR sequences may be used or exchanged for any of the UTR sequences described herein. UTRs may also be omitted from the mRNA polynucleotides provided herein.

Nucleic acids comprise a polymer of nucleotides (nucleotide monomers). Thus, nucleic acids are also referred to as polynucleotides. Nucleic acids may be or may include, for example, deoxyribonucleic acids (DNAs), ribonucleic acids (RNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) and/or chimeras and/or combinations thereof.

Messenger RNA (mRNA) is any RNA that encodes a (at least one) protein (a naturally occurring, non-naturally occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents mRNA, the “T”'s would be substituted for “U”s. Thus, any of the DNAs disclosed and identified by a particular sequence identification number herein also disclose the corresponding mRNA sequence complementary to the DNA, where each “T” of the DNA sequence is substituted with “U.”

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and 3′ UTRs, but that those elements, unlike the ORF, need not necessarily be present in an mRNA polynucleotide of the present disclosure.

### Variants

In some embodiments, the vaccines of the present disclosure include mRNA that encodes an EBV antigen variant. Antigen variants or other polypeptide variants refers to molecules that differ in their amino acid sequence from a wild-type, native, or reference sequence. The antigen/polypeptide variants may possess substitutions, deletions, and/or insertions at certain positions within the amino acid sequence, as compared to a native or reference sequence. Ordinarily, variants possess at least 50% identity to a wild-type, native or reference sequence. In some embodiments, variants share at least 80%, at least 85%, or at least 90% identity with a wild-type, native, or reference sequence.

Variant antigens/polypeptides encoded by nucleic acids of the disclosure may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property. Assays to determine expression levels and immunogenicity are well known in the art and exemplary such assays are set forth in the Examples section. Similarly, PK/PD properties of a protein variant can be measured using art recognized techniques, e.g., by determining expression of antigens in a vaccinated subject over time and/or by looking at the durability of the induced immune response. The stability of protein(s) encoded by a variant nucleic acid may be measured by assaying thermal stability or stability upon urea denaturation or may be measured using in silico prediction. Methods for such experiments and in silico determinations are known in the art.

In some embodiments, a vaccine comprises an mRNA or an mRNA open reading frame that comprises a nucleotide sequence of any one of the sequences provided herein (see, e.g., Sequence Listing and Table 3), or comprises a nucleotide sequence at least 80%, at least 85%, at least 90%, at least 95%, at least 96%, at least 97%, at least 98%, or at least 99% identical to a nucleotide sequence of any one of the sequences provided herein.

The term “identity” refers to a relationship between the sequences of two or more polypeptides (e.g., antigens) or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related antigens or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide (e.g., antigen) have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm.

As such, polynucleotides encoding peptides or polypeptides containing substitutions, insertions and/or additions, deletions and covalent modifications with respect to reference sequences, in particular the polypeptide (e.g., antigen) sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability.

In yet other embodiments, glycosylation sites may be removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an mRNA vaccine.

As recognized by those skilled in the art, protein fragments, functional protein domains, and homologous proteins are also considered to be within the scope of EBV antigens of interest. For example, provided herein is any protein fragment (meaning a polypeptide sequence at least one amino acid residue shorter than a reference antigen sequence but otherwise identical) of a reference protein, provided that the fragment is immunogenic and confers a protective immune response to the EBV. In addition to variants that are identical to the reference protein but are truncated, in some embodiments, an antigen includes 2, 3, 4, 5, 6, 7, 8, 9, 10, or more mutations, as shown in any of the sequences provided or referenced herein. Antigens/antigenic polypeptides can range in length from about 4, 6, or 8 amino acids to full length proteins.

### Stabilizing Elements

Naturally occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, a vaccine includes an mRNA having an open reading frame encoding at least one antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, MA). 5′-capping of mRNA may be completed post-transcriptionally using a Vaccinia Virus Capping Enzyme to generate the “Cap 0” structure: m7G(5′)ppp(5′)G (New England BioLabs, Ipswich, MA). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, a vaccine includes a stabilizing element. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The mRNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, an mRNA includes a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine:guanine phosphoribosyl transferase (GPT)).

In some embodiments, an mRNA includes the combination of a poly(A) sequence or polyadenylation signal and at least one histone stem-loop, even though both represent alternative mechanisms in nature, acts synergistically to increase the protein expression beyond the level observed with either of the individual elements. The synergistic effect of the combination of poly(A) and at least one histone stem-loop does not depend on the order of the elements or the length of the poly(A) sequence.

In some embodiments, an mRNA does not include a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. In some embodiments, the nucleic acid does not include an intron.

An mRNA may or may not contain an enhancer and/or promoter sequence, which may be modified or unmodified or which may be activated or inactivated. In some embodiments, the histone stem-loop is generally derived from histone genes and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures but may be present in single-stranded DNA as well. Stability of the stem-loop structure generally depends on the length, number of mismatches or bulges, and base vaccine of the paired region. In some embodiments, wobble base pairing (non-Watson-Crick base pairing) may result. In some embodiments, the at least one histone stem-loop sequence comprises a length of 15 to 45 nucleotides.

In some embodiments, an mRNA has one or more AU-rich sequences removed. These sequences, sometimes referred to as AURES are destabilizing sequences found in the 3′UTR. The AURES may be removed from the mRNA vaccines. Alternatively, the AURES may remain in the mRNA vaccine.

### Signal Peptides

In some embodiments, a vaccine comprises an mRNA having an open reading frame that encodes a signal peptide fused to the EBV antigen. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by a ER-resident signal peptidase of the host cell, or they remain uncleaved and function as a membrane anchor. A signal peptide may also facilitate the targeting of the protein to the cell membrane.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than EBV antigens in nature) are known in the art and can be tested for desired properties and then incorporated into a nucleic acid of the disclosure. In some embodiments, the signal peptide may comprise one of the following sequences: MDSKGSSQKGSRLLLLLVVSNLLLPQGVVG (SEQ ID NO: 26), MDWTWILFLVAAATRVHS (SEQ ID NO: 27); METPAQLLFLLLLWLPDTTG (SEQ ID NO: 28); MLGSNSGQRVVFTILLLLVAPAYS (SEQ ID NO: 29); MKCLLYLAFLFIGVNCA (SEQ ID NO: 30); MWLVSLAIVTACAGA (SEQ ID NO: 31).

### Fusion Proteins

In some embodiments, a vaccine of the present disclosure includes an mRNA encoding an antigenic fusion protein. Thus, the encoded antigen or antigens may include two or more proteins (e.g., protein and/or protein fragment) joined together. Alternatively, the protein to which a protein antigen is fused does not promote a strong immune response to itself, but rather to the EBV antigen. Antigenic fusion proteins, in some embodiments, retain the functional property from each original protein.

### Scaffold Moieties

The mRNA vaccines as provided herein, in some embodiments, encode fusion proteins that comprise EBV antigens linked to scaffold moieties. In some embodiments, such scaffold moieties impart desired properties to an antigen encoded by a nucleic acid of the disclosure. For example, scaffold proteins may improve the immunogenicity of an antigen, e.g., by altering the structure of the antigen, altering the uptake and processing of the antigen, and/or causing the antigen to bind to a binding partner.

In some embodiments, the scaffold moiety is protein that can self-assemble into protein nanoparticles that are highly symmetric, stable, and structurally organized, with diameters of 10-150 nm, a highly suitable size range for optimal interactions with various cells of the immune system. In some embodiments, viral proteins or virus-like particles can be used to form stable nanoparticle structures. Examples of such viral proteins are known in the art. For example, in some embodiments, the scaffold moiety is a hepatitis B surface antigen (HBsAg). HBsAg forms spherical particles with an average diameter of ˜22 nm and which lacked nucleic acid and hence are non-infectious (Lopez-Sagaseta, J. et al. Computational and Structural Biotechnology Journal 14 (2016) 58-68). In some embodiments, the scaffold moiety is a hepatitis B core antigen (HBcAg) self-assembles into particles of 24-31 nm diameter, which resembled the viral cores obtained from HBV-infected human liver. HBcAg produced in self-assembles into two classes of differently sized nanoparticles of 300 Å and 360 Å diameter, corresponding to 180 or 240 protomers. In some embodiments, the EBV antigen is fused to HBSAG or HBcAG to facilitate self-assembly of nanoparticles displaying the EBV antigen.

In some embodiments, bacterial protein platforms may be used. Non-limiting examples of these self-assembling proteins include ferritin, lumazine and encapsulin.

Ferritin is a protein whose main function is intracellular iron storage. Ferritin is made of 24 subunits, each composed of a four-alpha-helix bundle, that self-assemble in a quaternary structure with octahedral symmetry (Cho K. J. et al. J Mol Biol. 2009; 390:83-98). Several high-resolution structures of ferritin have been determined, confirming that Helicobacter pylori ferritin is made of 24 identical protomers, whereas in animals, there are ferritin light and heavy chains that can assemble alone or combine with different ratios into particles of 24 subunits (Granier T. et al. J Biol Inorg Chem. 2003; 8:105-111; Lawson D. M. et al. Nature. 1991; 349:541-544). Ferritin self-assembles into nanoparticles with robust thermal and chemical stability. Thus, the ferritin nanoparticle is well-suited to carry and expose antigens.

Lumazine synthase (LS) is also well-suited as a nanoparticle platform for antigen display. LS, which is responsible for the penultimate catalytic step in the biosynthesis of riboflavin, is an enzyme present in a broad variety of organisms, including archaea, bacteria, fungi, plants, and eubacteria (Weber S. E. Flavins and Flavoproteins. Methods and Protocols, Series: Methods in Molecular Biology. 2014). The LS monomer is 150 amino acids long and consists of beta-sheets along with tandem alpha-helices flanking its sides. A number of different quaternary structures have been reported for LS, illustrating its morphological versatility: from homopentamers up to symmetrical assemblies of 12 pentamers forming capsids of 150 Å diameter. Even LS cages of more than 100 subunits have been described (Zhang X. et al. J Mol Biol. 2006; 362:753-770).

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers having a thin and icosahedral T=1 symmetric cage structure with interior and exterior diameters of 20 and 24 nm, respectively (Sutter M. et al. Nat Struct Mol Biol. 2008, 15: 939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses (Rahmanpour R. et al. FEBS J. 2013, 280: 2097-2104).

### Linkers and Cleavable Peptides

In some embodiments, the mRNAs of the disclosure encode more than one polypeptide, referred to herein as fusion proteins. In some embodiments, the mRNA further encodes a linker located between at least one or each domain of the fusion protein. The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2A peptides, has been described in the art (see for example, Kim, J. H. et al. (2011) PLOS ONE 6:018556). In some embodiments, the linker is an F2A linker. In some embodiments, the linker is a GGGS linker. In some embodiments, the fusion protein contains three domains with intervening linkers, having the structure: domain-linker-domain-linker-domain.

Cleavable linkers known in the art may be used in connection with the disclosure. Exemplary such linkers include: F2A linkers, T2A linkers, P2A linkers, E2A linkers (See, e.g., WO2017127750). The skilled artisan will appreciate that other art-recognized linkers may be suitable for use in the mRNAs of the disclosure (e.g., encoded by the nucleic acids of the disclosure). The skilled artisan will likewise appreciate that other polycistronic mRNA encoding more than one antigen/polypeptide separately within the same molecule) may be suitable for use as provided herein.

### Sequence Optimization

In some embodiments, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally occurring or wild-type sequence ORF (e.g., a naturally occurring or wild-type mRNA sequence encoding an EBV antigen). In some embodiments, a codon optimized sequence shares less than 90% sequence identity to a naturally occurring or wild-type sequence (e.g., a naturally occurring or wild-type mRNA sequence encoding an EBV antigen). In some embodiments, a codon optimized sequence shares less than 85% sequence identity to a naturally occurring or wild-type sequence (e.g., a naturally occurring or wild-type mRNA sequence encoding an EBV antigen). In some embodiments, a codon optimized sequence shares less than 80% sequence identity to a naturally occurring or wild-type sequence (e.g., a naturally occurring or wild-type mRNA sequence encoding an EBV antigen). In some embodiments, a codon optimized sequence shares less than 75% sequence identity to a naturally occurring or wild-type sequence (e.g., a naturally occurring or wild-type mRNA sequence encoding an EBV antigen).

In some embodiments, a codon optimized sequence shares between 65% and 85% (e.g., between about 67% and about 85% or between about 67% and about 80%) sequence identity to a naturally occurring or wild-type sequence (e.g., a naturally occurring or wild-type mRNA sequence encoding an EBV antigen). In some embodiments, a codon optimized sequence shares between 65% and 75% or about 80% sequence identity to a naturally occurring or wild-type sequence (e.g., a naturally occurring or wild-type mRNA sequence encoding an EBV antigen).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than an EBV antigen encoded by a non-codon-optimized sequence.

When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized mRNA may be one in which the levels of G/C are enhanced. The G/C-content of nucleic acid molecules (e.g., mRNA) may influence the stability of the mRNA. RNA having an increased amount of guanine (G) and/or cytosine (C) residues may be functionally more stable than mRNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater mRNA stability without changing the resulting amino acid. The approach is limited to coding regions of the mRNA.

### Chemically Unmodified Nucleotides

In some embodiments, an mRNA is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed mRNA (e.g., A, G, C, or U). In some embodiments, nucleotides and nucleosides of the present disclosure comprise standard deoxyribonucleosides such as those present in DNA (e.g., dA, dG, dC, or dT).

### Chemical Modifications

The vaccines of the present disclosure comprise, in some embodiments, an mRNA having an open reading frame encoding an EBV antigen, wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published US application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB2017/051367 all of which are incorporated by reference herein.

Hence, nucleic acids of the disclosure (e.g., DNA nucleic acids and RNA nucleic acids, such as mRNA nucleic acids) can comprise standard nucleotides and nucleosides, naturally occurring nucleotides and nucleosides, non-naturally occurring nucleotides and nucleosides, or any combination thereof.

Nucleic acids of the disclosure (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified mRNA, introduced to a cell or organism, exhibits reduced degradation in the cell or organism, respectively, relative to an unmodified nucleic acid comprising standard nucleotides and nucleosides.

In some embodiments, a modified mRNA, introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise 1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a mRNA of the disclosure comprises 1-methyl-pseudouridine (m1ψ) substitutions at one or more or all uridine positions of the nucleic acid.

In some embodiments, a mRNA of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a mRNA of the disclosure comprises pseudouridine (ψ) substitutions at one or more or all uridine positions of the nucleic acid.

In some embodiments, a mRNA of the disclosure comprises pseudouridine (ψ) substitutions at one or more or all uridine positions of the nucleic acid and 5-methyl cytidine substitutions at one or more or all cytidine positions of the nucleic acid.

In some embodiments, a mRNA of the disclosure comprises uridine at one or more or all uridine positions of the nucleic acid.

In some embodiments, mRNAs are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridin. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A. G. U. C) may be uniformly modified in a nucleic acid of the disclosure, or in a predetermined sequence region thereof (e.g., in the mRNA including or excluding the poly(A) tail). In some embodiments, all nucleotides X in a nucleic acid of the present disclosure (or in a sequence region thereof) are modified nucleotides, wherein X may be any one of nucleotides A, G. U. C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A. G. U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The mRNAs may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure or can be replaced by a plurality of compounds having different structures (e.g., 2, 3, 4 or more unique structures).

### Untranslated Regions (UTRs)

The mRNAs of the present disclosure may comprise one or more regions or parts which act or function as an untranslated region. Where mRNAs are designed to encode at least one antigen of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′ UTR and 3′ UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding). Natural 5′ UTRs have features that play roles in translation initiation. They harbor signatures like Kozak sequences which are commonly known to be involved in the process by which the ribosome initiates translation of many genes. Kozak sequences have the consensus CCR(A/G)CCAUGG (SEQ ID NO: 32), where R is a purine (adenine or guanine) three bases upstream of the start codon (AUG), which is followed by another ‘G’. 5′ UTRs also have been known to form secondary structures which are involved in elongation factor binding.

In some embodiments of the disclosure, a 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219). CMV immediate-early 1 (IE1) gene (US20140206753, WO2013/185069), the sequence GGGAUCCUACC (SEQ ID NO: 33) (WO2014144196) may also be used. In another embodiment, 5′ UTR of a TOP gene is a 5′ UTR of a TOP gene lacking the 5′ TOP motif (the oligopyrimidine tract) (e.g., WO/2015101414, WO2015101415, WO/2015/062738, WO2015024667, WO2015024667; 5′ UTR element derived from ribosomal protein Large 32 (L32) gene (WO/2015101414, WO2015101415, WO/2015/062738), 5′ UTR element derived from the 5′UTR of an hydroxysteroid (17-β) dehydrogenase 4 gene (HSD17B4) (WO2015024667), or a 5′ UTR element derived from the 5′ UTR of ATP5A1 (WO2015024667) can be used. In some embodiments, an internal ribosome entry site (IRES) is used instead of a 5′ UTR.

In some embodiments, a 5′ UTR of the present disclosure comprises a sequence selected from SEQ ID NO: 1 and SEQ ID NO: 24.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) (SEQ ID NO: 34) nonamers. Molecules containing this type of AREs include GM-CSF and TNF-a. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of nucleic acids (e.g., mRNA) of the disclosure. When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hours, 12 hours, 24 hours, 48 hours, 3 days, 4 days, 5 days, 6 days, and/or 7 days post-transfection.

3′ UTRs may be heterologous or synthetic. With respect to 3′ UTRs, globin UTRs, including Xenopus β-globin UTRs and human β-globin UTRs are known in the art (U.S. Pat. Nos. 8,278,063, 9,012,219, US20110086907). A nucleic acid (e.g., mRNA) encoding a modified β-globin with enhanced stability in some cell types by cloning two sequential human β-globin 3′ UTRs head to tail has been developed and is well known in the art (US2012/0195936, WO2014/071963). In addition, a2-globin, al-globin, UTRs and mutants thereof are also known in the art

(WO2015101415, WO2015024667). Other 3′ UTRs described in the mRNA in the non-patent literature include CYBA (Ferizi et al., 2015) and albumin (Thess et al., 2015). Other exemplary 3′ UTRs include that of bovine or human growth hormone (wild type or modified) (WO2013/185069, US20140206753, WO2014152774), rabbit β globin and hepatitis B virus (HBV), α-globin 3′ UTR and Viral VEEV 3′ UTR sequences are also known in the art. In some embodiments, the sequence UUUGAAUU (WO2014144196) is used. In some embodiments, 3′ UTRs of human and mouse ribosomal protein are used. Other examples include rps9 3′ UTR (WO2015101414), FIG. 4 (WO2015101415), and human albumin 7 (WO2015101415).

In some embodiments, a 3′ UTR of the present disclosure comprises a sequence selected from SEQ ID NO: 3 and SEQ ID NO: 25.

Those of ordinary skill in the art will understand that 5′ UTRs that are heterologous or synthetic may be used with any desired 3′ UTR sequence. For example, a heterologous 5′ UTR may be used with a synthetic 3′ UTR with a heterologous 3′ UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No. 20100293625 and PCT/US2014/069155, herein incorporated by reference in its entirety.

It should be understood that any UTR from any gene may be incorporated into the regions of a nucleic acid. Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 20100129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in US Application No. 20090226470, herein incorporated by reference in its entirety, and those known in the art.

In vitro Transcription of mRNA cDNA encoding the polynucleotides described herein may be transcribed using an in vitro transcription (IVT) system. In vitro transcription of mRNA is known in the art and is described in International Publication WO/2014/152027, which is incorporated by reference herein in its entirety.

In some embodiments, the mRNA transcript is generated using a non-amplified, linearized DNA template in an in vitro transcription reaction to generate the mRNA transcript. In some embodiments, the template DNA is isolated DNA. In some embodiments, the template DNA is cDNA. In some embodiments, the cDNA is formed by reverse transcription of a mRNA, for example, but not limited to EBV mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes an RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a poly(A) tail. The particular nucleic acid sequence and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide. When mRNA transcripts are being generated, the 5′ UTR may comprise a promoter sequence. Such promoter sequences are known in the art. It should be understood that such promoter sequences will not be present in a vaccine of the disclosure.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “poly(A) tail” is a region of mRNA that is downstream, e.g., directly downstream (i.e., 3′), from the 3′ UTR that contains multiple, consecutive adenosine monophosphates. A poly(A) tail may contain 10 to 300 adenosine monophosphates. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly(A) tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a nucleic acid includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or 5 mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the mRNA transcript is capped via enzymatic capping. In some embodiments, the mRNA comprises 5′ terminal cap, for example, 7mG(5′)ppp(5′)NlmpNp.

### Chemical Synthesis

Solid-phase chemical synthesis. Nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

Liquid Phase Chemical Synthesis. The synthesis of nucleic acids of the present disclosure by the sequential addition of monomer building blocks may be carried out in a liquid phase.

Combination of Synthetic Methods. The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNA™ oligo-T capture probes (EXIQON® Inc, Vedback, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the nucleic acids of the present disclosure may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using antigen-specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunosorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Lipid Nanoparticles (LNPs)

In some embodiments, the mRNA of the disclosure is formulated in a lipid nanoparticle

(LNP). Lipid nanoparticles typically comprise ionizable amino lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016/000129; PCT/US2016/014280; PCT/US2016/014280; PCT/US2017/038426; PCT/US2014/027077; PCT/US2014/055394; PCT/US2016/052117; PCT/US2012/069610; PCT/US2017/027492; PCT/US2016/059575 and PCT/US2016/069491 all of which are incorporated by reference herein in their entirety.

Vaccines of the present disclosure are typically formulated in lipid nanoparticles. The vaccines can be made, for example, using mixing processes such as microfluidics and T-junction mixing of two fluid streams, one of which contains the mRNA and the other has the lipid components. In some embodiments, the vaccines are prepared by combining an ionizable amino lipid, a phospholipid (such as DOPE or DSPC), a PEG lipid (such as 1,2-dimyristoyl-OT-glycerol methoxypoly ethylene glycol, also known as PEG-DMG), and a structural lipid (such as cholesterol) in an alcohol (e.g., ethanol). The lipids may be combined to yield desired molar ratios and diluted with water and alcohol (e.g., ethanol) to a final lipid concentration of between about 5.5 mM and about 25 mM, for example.

Vaccines including mRNA and a lipid component may be prepared, for example, by combining a lipid solution with an mRNA solution at lipid component to mRNA wt:wt ratios of between about 5:1 and about 50:1. The lipid solution may be rapidly injected using a microfluidic based system (e.g., NanoAssemblr) at flow rates between about 10 ml/min and about 18 ml/min, for example, into the mRNA solution to produce a suspension (e.g., with a water to alcohol ratio between about 1:1 and about 4:1).

Vaccines can be processed by dialysis to remove the alcohol (e.g., ethanol) and achieve buffer exchange. Formulations may be dialyzed against phosphate buffered saline (PBS), pH 7.4, for example, at volumes greater than that of the primary product (e.g., using Slide-A-Lyzer cassettes (Thermo Fisher Scientific Inc., Rockford, IL)) with a molecular weight cutoff of 10 kD, for example. The forgoing exemplary method induces nanoprecipitation and particle formation. Alternative processes including, but not limited to, T-junction and direct injection, may be used to achieve the same nanoprecipitation.

In some embodiments, the lipid nanoparticle comprises at least one ionizable amino (cationic) lipid, at least one non-cationic lipid, at least one sterol, and/or at least one polyethylene glycol (PEG)-modified lipid.

In some embodiments, the lipid nanoparticle comprises 20-60 mol % ionizable amino lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, the lipid nanoparticle comprises 20 mol %, 30 mol %, 40 mol %, 50, or 60 mol % ionizable amino lipid.

In some embodiments, the lipid nanoparticle comprises 5-25 mol % non-cationic lipid. For example, the lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, the lipid nanoparticle comprises 5 mol %, 10 mol %, 15 mol %, 20 mol %, or 25 mol % non-cationic lipid.

In some embodiments, the lipid nanoparticle comprises 25-55 mol % sterol. For example, the lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid nanoparticle comprises 25 mol %, 30 mol %, 35 mol %, 40 mol %, 45 mol %, 50 mol %, or 55 mol % sterol.

In some embodiments, the lipid nanoparticle comprises 0.5-15 mol % PEG-modified lipid. For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %. In some embodiments, the lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

In some embodiments, the lipid nanoparticle comprises 20-60 mol % ionizable amino lipid, 5-25 mol % non-cationic lipid, 25-55 mol % sterol, and 0.5-15 mol % PEG-modified lipid.

In some embodiments, an ionizable amino lipid of the disclosure comprises a compound of Formula (I):

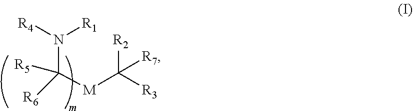

or a salt or isomer thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

In some embodiments, a subset of compounds of Formula (I) includes those in which when R₄ is —(CH₂)_{b}Q, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR. —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂. —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13, or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13, or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13, or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13, or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13, or salts or isomers thereof.

In some embodiments, a subset of compounds of Formula (I) includes those of Formula (IA):

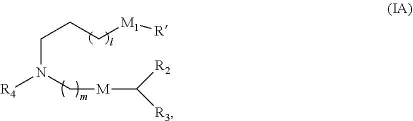

or a salt or isomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (II):

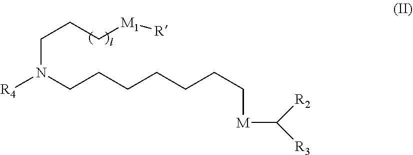

or a salt or isomer thereof, wherein 1 is selected from 1, 2, 3, 4, and 5; M₁ is a bond or M′; R₄ is unsubstituted C₁₋₃ alkyl, or —(CH₂)»Q, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (IIa), (IIb), (IIc), or (IIe):

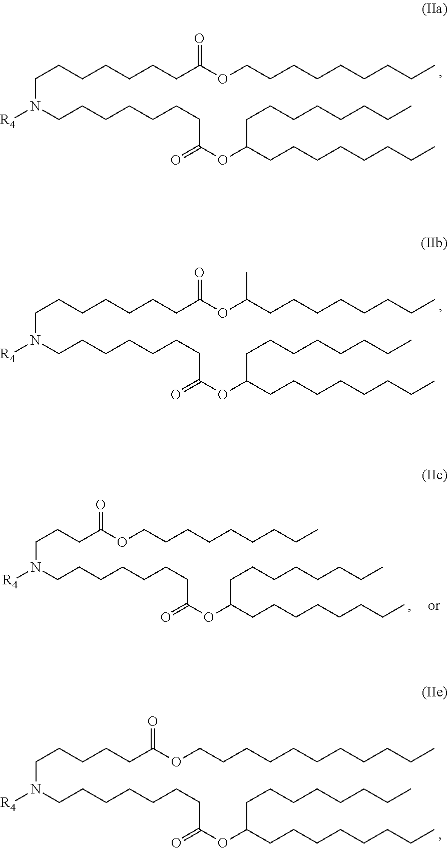

or a salt or isomer thereof, wherein R₄ is as described herein.

In some embodiments, a subset of compounds of Formula (I) includes those of Formula (IId):

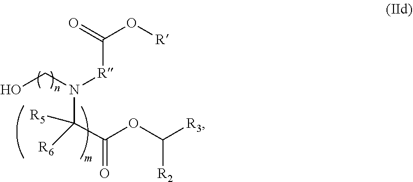

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable amino lipid of the disclosure comprises a compound having structure:

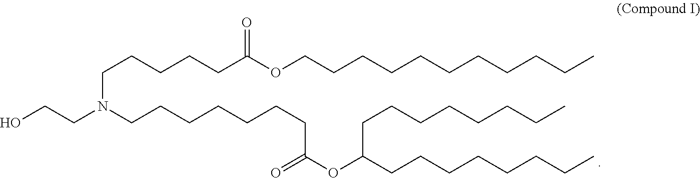 [SMILES: CCCCCCCCCCCOC(=O)CCCCCN(CCO)CCCCCCCC(=O)OC(CCCCCCCC)CCCCCCCC]

In some embodiments, an ionizable amino lipid of the disclosure comprises a compound having structure:

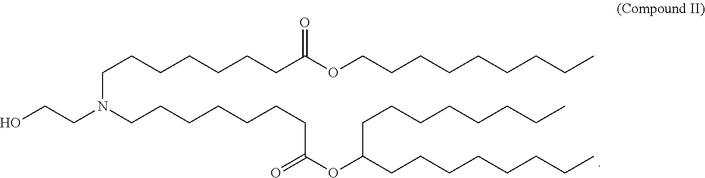 [SMILES: CCCCCCCCCOC(=O)CCCCCCCN(CCO)CCCCCCCC(=O)OC(CCCCCCCC)CCCCCCCC]

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-diolcoyl-sn-glycero-3-phospho-rac-(1-glycerol) sodium salt (DOPG), sphingomyelin, and mixtures thereof.

In some embodiments, a PEG modified lipid of the disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is DMG-PEG, PEG-c-DOMG (also referred to as PEG-DOMG), PEG-DSG and/or PEG-DPG.

In some embodiments, a sterol of the disclosure comprises cholesterol, fecosterol, sitosterol, ergosterol, campesterol, stigmasterol, brassicasterol, tomatidine, ursolic acid, alpha-tocopherol, and mixtures thereof.

In some embodiments, a LNP of the disclosure comprises an ionizable amino lipid of Compound 1, wherein the non-cationic lipid is DSPC, the structural lipid that is cholesterol, and the PEG lipid is DMG-PEG.

In some embodiments, the lipid nanoparticle comprises 45-55 mole percent ionizable amino lipid. For example, lipid nanoparticle may comprise 45, 46, 47, 48, 49, 50, 51, 52, 53, 54, or 55 mole percent ionizable amino lipid.

In some embodiments, the lipid nanoparticle comprises 5-15 mole percent DSPC. For example, the lipid nanoparticle may comprise 5, 6, 7, 8, 9, 10, 11, 12, 13, 14, or 15 mole percent DSPC.

In some embodiments, the lipid nanoparticle comprises 35-40 mole percent cholesterol. For example, the lipid nanoparticle may comprise 35, 36, 37, 38, 39, or 40 mole percent cholesterol.

In some embodiments, the lipid nanoparticle comprises 1-2 mole percent DMG-PEG. For example, the lipid nanoparticle may comprise 1, 1.5, or 2 mole percent DMG-PEG.

In some embodiments, the lipid nanoparticle comprises 50 mole percent ionizable amino lipid, 10 mole percent DSPC, 38.5 mole percent cholesterol, and 1.5 mole percent DMG-PEG.

In some embodiments, a LNP of the disclosure comprises an N:P ratio of from about 2:1 to about 30:1.

In some embodiments, a LNP of the disclosure comprises an N:P ratio of about 6:1.

In some embodiments, a LNP of the disclosure comprises an N:P ratio of about 3:1.

In some embodiments, a LNP of the disclosure comprises a wt/wt ratio of the ionizable amino lipid component to the mRNA of from about 10:1 to about 100:1.

In some embodiments, a LNP of the disclosure comprises a wt/wt ratio of the ionizable amino lipid component to the mRNA of about 20:1.

In some embodiments, a LNP of the disclosure comprises a wt/wt ratio of the ionizable amino lipid component to the mRNA of about 10:1.

In some embodiments, a LNP of the disclosure has a mean diameter from about 50 nm to about 150 nm.

In some embodiments, a LNP of the disclosure has a mean diameter from about 70 nm to about 120 nm.

### Multivalent Vaccines

The vaccines, as provided herein, include multiple mRNAs, each encoding one or more EBV antigens. In some embodiments, a vaccine includes 1, 2, 3, 4, 5, 6, 7, 8, 9, 10, 11, 12, or more mRNAs encoding EBV antigens.

In some embodiments, two or more different mRNA encoding antigens may be formulated in the same lipid nanoparticle. In other embodiments, two or more different mRNA encoding antigens may be formulated in separate lipid nanoparticles (each mRNA formulated in a single lipid nanoparticle). The lipid nanoparticles may then be combined and administered as a single vaccine (e.g., comprising multiple mRNA encoding multiple antigens) or may be administered separately.

In some embodiments, the EBV vaccines provided herein include a first mRNA encoding EBV gp220, a second mRNA encoding EBV gp42 (soluble or wild type), a third mRNA encoding EBV gH, and a fourth mRNA encoding EBV gL. In some embodiments, the EBV vaccine further comprises a fifth mRNA encoding EBV gB.

Multivalent mRNAs are typically produced by transcribing one mRNA at a time, purifying each mRNA, and then mixing the purified mRNA together prior to formulation.

In some embodiments, a vaccine comprises (a) a first mRNA comprising an open reading frame encoding EBV glycoprotein 220 (gp220); (b) a second mRNA comprising an open reading frame encoding glycoprotein 42 (gp42) (e.g., a soluble form of gp42); (c) a third mRNA comprising an open reading frame encoding glycoprotein L (gL); and (d) a fourth mRNA comprising an open reading frame encoding glycoprotein H (gH).

In some embodiments, the mass ratio of (a):(b) is 4:1, 4:1.5, 2:1, 4:2.5, 4:3, 4:3.5, or 1:1. In some embodiments, the mass ratio of (a):(c) 4:1, 4:1.5, 2:1, 4:2.5, 4:3, 4:3.5, or 1:1. In some embodiments, the mass ratio of (a):(d) 4:1, 4:1.5, 2:1, 4:2.5, 4:3, 4:3.5, or 1:1.

In some embodiments, the mass ratio of (b):(c) is 2:1, 2:1.5, 1:1, 1:1.5, or 1:2. In some embodiments, the ratio of (b):(d) is 2:1, 2:1.5, 1:1, 1:1.5, or 1:2.

In some embodiments, the mass ratio of (c):(d) is 2:1, 2:1.5, 1:1, 1:1.5, or 1:2.

In some embodiments, the mass ratio of (a):(b):(c):(d) is 4:1:1:1.5. In some embodiments, the mass ratio of (a):(b):(c):(d) is 4:1:1:1.

In some embodiments, a vaccine comprises mRNA encoding EBV gp220 at 5×, 4.5×, 4×, 3.5×, 3×, 2.5×, 2×, or 1.5× the mRNA encoding EBV gp42. In some embodiments, a vaccine comprises mRNA encoding EBV gp220 at equal mass to the mRNA encoding EBV gp42.

In some embodiments, a vaccine comprises mRNA encoding EBV gp220 at 5×, 4.5×, 4×, 3.5×, 3×, 2.5×, 2×, or 1.5× the mRNA encoding EBV gL. In some embodiments, a vaccine comprises mRNA encoding EBV gp220 at equal mass to the mRNA encoding EBV gL.

In some embodiments, a vaccine comprises mRNA encoding EBV gp220 at 5×, 4.5×, 4×, 3.5×, 3×, 2.5×, 2×, or 1.5× the mRNA encoding EBV gH. In some embodiments, a vaccine comprises mRNA encoding EBV gp220 at equal mass to the mRNA encoding EBV gH.

In some embodiments, a vaccine comprises mRNA encoding EBV gp42 at 5×, 4.5×, 4×, 3.5×, 3×, 2.5×, 2×, or 1.5× the mRNA encoding EBV gp220. In some embodiments, a vaccine comprises mRNA encoding EBV gp42 at equal mass to the mRNA encoding EBV gp220.

In some embodiments, a vaccine comprises mRNA encoding EBV gp42 at 5×, 4.5×, 4×, 3.5×, 3×, 2.5×, 2×, or 1.5× the mRNA encoding EBV gL. In some embodiments, a vaccine comprises mRNA encoding EBV gp42 at equal mass to the mRNA encoding EBV gL.

In some embodiments, a vaccine comprises mRNA encoding EBV gp42 at 5×, 4.5×, 4×, 3.5×, 3×, 2.5×, 2×, or 1.5× the mRNA encoding EBV gH. In some embodiments, a vaccine comprises mRNA encoding EBV gp42 at equal mass to the mRNA encoding EBV gH.

In some embodiments, a vaccine comprises mRNA encoding EBV gL at 5×, 4.5×, 4×, 3.5×, 3×, 2.5×, 2×, or 1.5× the mRNA encoding EBV gp220. In some embodiments, a vaccine comprises mRNA encoding EBV gL at equal mass to the mRNA encoding EBV gp220.

In some embodiments, a vaccine comprises mRNA encoding EBV gL at 5×, 4.5×, 4×, 3.5×, 3×, 2.5×, 2×, or 1.5× the mRNA encoding EBV gp42. In some embodiments, a vaccine comprises mRNA encoding EBV gL at equal mass to the mRNA encoding EBV g42.

In some embodiments, a vaccine comprises mRNA encoding EBV gL at 5×, 4.5×, 4×, 3.5×, 3×, 2.5×, 2×, or 1.5× the mRNA encoding EBV gH. In some embodiments, a vaccine comprises mRNA encoding EBV gL at equal mass to the mRNA encoding EBV gH.

In some embodiments, a vaccine comprises mRNA encoding EBV gH at 5×, 4.5×, 4×, 3.5×, 3×, 2.5×, 2×, or 1.5× the mRNA encoding EBV gp220. In some embodiments, a vaccine comprises mRNA encoding EBV gH at equal mass to the mRNA encoding EBV gp220.

In some embodiments, a vaccine comprises mRNA encoding EBV gH at 5×, 4.5×, 4×, 3.5×, 3×, 2.5×, 2×, or 1.5× the mRNA encoding EBV gp42. In some embodiments, a vaccine comprises mRNA encoding EBV gH at equal mass to the mRNA encoding EBV gp42.

In some embodiments, a vaccine comprises mRNA encoding EBV gH at 5×, 4.5×, 4×, 3.5×, 3×, 2.5×, 2×, or 1.5× the mRNA encoding EBV gL. In some embodiments, a vaccine comprises mRNA encoding EBV gH at equal mass to the mRNA encoding EBV gL.

In some embodiments, the vaccine further comprises (c) a fifth mRNA comprising an open reading frame encoding EBV glycoprotein B (gB).

In some embodiments, the mass ratio of (a):(c) is 4:1, 4:1.5, 2:1, 4:2.5, 4:3, 4:3.5, or 1:1. In some embodiments, the mass ratio of (b):(c) is 2:1, 2:1.5, 1:1, 1:1.5, or 1:2. In some embodiments, the ratio of (c):(c) is 2:1, 2:1.5, 1:1, 1:1.5, or 1:2. In some embodiments, the mass ratio of (d):(c) is 2:1, 2:1.5, 1:1, 1:1.5, or 1:2.

In some embodiments, the mass ratio of (a):(b):(c):(d):(c) is 4:1:1:1.5:1.5. In some embodiments, the ratio of (a):(b):(c):(d):(c) is 4:1:1:1:1.

In some embodiments, a vaccine comprises mRNA encoding EBV gp220 at 5×, 4.5×, 4×, 3.5×, 3×, 2.5×, 2×, or 1.5× the mRNA encoding EBV gB. In some embodiments, a vaccine comprises mRNA encoding EBV gp220 at equal mass to the mRNA encoding EBV gB.

In some embodiments, a vaccine comprises mRNA encoding EBV gp42 at 5×, 4.5×, 4×, 3.5×, 3×, 2.5×, 2×, or 1.5× the mRNA encoding EBV gB. In some embodiments, a vaccine comprises mRNA encoding EBV gp42 at equal mass to the mRNA encoding EBV gB.

In some embodiments, a vaccine comprises mRNA encoding EBV gL at 5×, 4.5×, 4×, 3.5×, 3×, 2.5×, 2×, or 1.5× the mRNA encoding EBV gB. In some embodiments, a vaccine comprises mRNA encoding EBV gL at equal mass to the mRNA encoding EBV gB.

In some embodiments, a vaccine comprises mRNA encoding EBV gH at 5×, 4.5×, 4×, 3.5×, 3×, 2.5×, 2×, or 1.5× the mRNA encoding EBV gB. In some embodiments, a vaccine comprises mRNA encoding EBV gH at equal mass to the mRNA encoding EBV gB.

In some embodiments, a vaccine comprises mRNA encoding EBV gB at 5×, 4.5×, 4×, 3.5×, 3×, 2.5×, 2×, or 1.5× the mRNA encoding EBV gp220. In some embodiments, a vaccine comprises mRNA encoding EBV gB at equal mass to the mRNA encoding EBV gp220.

In some embodiments, a vaccine comprises mRNA encoding EBV gB at 5×, 4.5×, 4×, 3.5×, 3×, 2.5×, 2×, or 1.5× the mRNA encoding EBV gp42. In some embodiments, a vaccine comprises mRNA encoding EBV gB at equal mass to the mRNA encoding EBV gp42.

In some embodiments, a vaccine comprises mRNA encoding EBV gB at 5×, 4.5×, 4×, 3.5×, 3×, 2.5×, 2×, or 1.5× the mRNA encoding EBV gL. In some embodiments, a vaccine comprises mRNA encoding EBV gB at equal mass to the mRNA encoding EBV gL.

In some embodiments, a vaccine comprises mRNA encoding EBV gB at 5×, 4.5×, 4×, 3.5×, 3×, 2.5×, 2×, or 1.5× the mRNA encoding EBV gH. In some embodiments, a vaccine comprises mRNA encoding EBV gB at equal mass to the mRNA encoding EBV gH.

### Vaccine Formulations and Dosing Schedules

Provided herein, in some aspects, are vaccines, methods, kits and reagents for prevention or treatment of EBV infection in humans. The vaccines provided herein can be used as prophylactic or therapeutic agents to prevent or treat EBV infection or disease progression. A vaccine may be administered prophylactically or therapeutically as part of an active immunization scheme to healthy individuals or early in infection during the incubation phase or during active infection after onset of symptoms. In some embodiments, the amount of mRNA provided to a cell, a tissue or a subject may be an amount effective for immune prophylaxis.

A vaccine may be utilized in various settings depending on the prevalence of the infection or the degree or level of unmet medical need. mRNA vaccines have superior properties in that they produce much larger antibody titers, better neutralizing immunity, produce more durable immune responses, and/or produce responses earlier than commercially available vaccines.

In some embodiments, the EBV vaccine containing mRNA as described herein can be administered to a subject (e.g., a mammalian subject, such as a human subject), and the mRNA is translated and expressed in vivo to produce an EBV antigen, which then stimulates an immune response in the subject. In some embodiments, an mRNA vaccine is administered to a subject (e.g., a mammalian subject, such as a human subject) in an effective amount to induce an antigen-specific (e.g., EBV-specific) immune response.

### Formulation/Dose

A vaccine comprising mRNA and LNP may or may not further comprise one or more other components. For example, a vaccine may include other components including, but not limited to, adjuvants and/or excipients. In some embodiments, a vaccine does not include an adjuvant (they are adjuvant free).

Vaccines may be sterile, pyrogen-free or both sterile and pyrogen-free. General considerations in the formulation and/or manufacture of pharmaceutical agents, such as vaccines, may be found, for example, in Remington: The Science and Practice of Pharmacy 21st ed., Lippincott Williams & Wilkins, 2005 (incorporated herein by reference in its entirety).

Formulations of the vaccines described herein may be prepared by any method known or hereafter developed in the art of pharmacology. In general, such preparatory methods include the step of bringing the active ingredient (e.g., mRNA) into association with an excipient and/or one or more other accessory ingredients (e.g., lipid nanoparticle components described herein), and then, if necessary and/or desirable, dividing, shaping and/or packaging the product into a desired single- or multi-dose unit.

Relative amounts of the active ingredient (i.e., mRNA), the pharmaceutically acceptable excipient (e.g., LNP components), and/or any additional ingredients in a vaccine in accordance with the disclosure will vary, depending upon the identity, size, and/or condition of the subject treated and further depending upon the route by which the vaccine is to be administered. By way of example, the vaccine may comprise between 0.1% and 100%, e.g., between 0.5 and 50%, between 1-30%, between 5-80%, at least 80% (w/w) mRNA.

In some embodiments, an RNA is formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with the mRNA (e.g., for transplantation into a subject), hyaluronidase, nanoparticle mimics and combinations thereof.

An effective amount (e.g., effective dose) prevents infection by the virus at a clinically acceptable level. In some embodiments, the effective dose is a dose listed in a package insert for the vaccine. An effective amount (e.g., effective dose) of a vaccine (e.g., comprising mRNA) is based, at least in part, on the target tissue, target cell type, means of administration, physical characteristics of the mRNA (e.g., length, nucleotide composition, and/or extent of modified nucleosides), other components of the vaccine, and other determinants, such as age, body weight, height, sex and general health of the subject. Typically, an effective amount of a vaccine provides an induced or boosted immune response as a function of antigen production in the cells of the subject. In some embodiments, an effective amount of the vaccine containing mRNA having at least one chemical modification is more efficient than a vaccine containing a corresponding unmodified polynucleotide encoding the same antigen or a peptide antigen. Increased antigen production may be demonstrated by increased cell transfection (the percentage of cells transfected with the mRNA vaccine), increased protein translation and/or expression from the polynucleotide, decreased nucleic acid degradation (as demonstrated, for example, by increased duration of protein translation from a modified polynucleotide), or altered antigen specific immune response of the host cell.

The effective amount of the mRNA, as provided herein, depends at least in part on the age of the subject being vaccinated and the vaccine dosing schedule. In some embodiments, the vaccine is administered as a single dose. In other some embodiments, one or more booster dose(s) of the vaccine is administered.

In some embodiments, the vaccine comprises about 25-125 μg mRNA encoding EBV gp220. For example, the vaccine may comprise about 25, 26, 27, 28, 29, 30, 35, 40, 45, 50, 51, 52, 53, 54, 55, 60, 65, 70, 75, 80, 85, 90, 95, 100, 105, 106, 107, 108, 109, 110, 115, 120, or 125 μg mRNA encoding EBV gp220. In some embodiments, the vaccine comprises about 25-30 μg. 50-55 μg, or 105-110 μg mRNA encoding EBV gp220.

In some embodiments, the vaccine comprises about 5-30 μg mRNA encoding EBV gp42. For example, the vaccine may comprise about 5, 6, 7, 8, 9, 10, 11, 12, 13, 14, 15, 16, 17, 18, 19, 20, 21, 22, 23, 24, 25, 26, 27, 28, 29, or 30 μg mRNA encoding EBV gp42. In some embodiments, the vaccine comprises about 5-10 μg. 10-15 μg, or 25-20 μg mRNA encoding EBV gp42.

In some embodiments, the vaccine comprises about 5-30 μg mRNA encoding EBV gL. For example, the vaccine may comprise about 5, 6, 7, 8, 9, 10, 11, 12, 13, 14, 15, 16, 17, 18, 19, 20, 21, 22, 23, 24, 25, 26, 27, 28, 29, or 30 μg mRNA encoding EBV gL. In some embodiments, the vaccine comprises about 5-10 μg. 10-15 μg, or 25-20 μg mRNA encoding EBV gL.

In some embodiments, the vaccine comprises about 10-50 μg mRNA encoding EBV gH. For example, the vaccine may comprise about 10, 15, 20, 25, 30, 35, 40, 45, or 50 μg mRNA encoding EBV gH. In some embodiments, the vaccine comprises about 5-15 μg. 15-25 μg, or 30-50 μg mRNA encoding EBV gH.

In some embodiments, the vaccine comprises 26.7 μg mRNA encoding EBV gp220, 6.7 μg mRNA encoding EBV gp42, 6.7 μg mRNA encoding EBV gL, and 10 μg mRNA encoding EBV gH.

In other embodiments, the vaccine comprises 53.3 μg mRNA encoding EBV gp220, 13.3 μg mRNA encoding EBV gp42, 13.3 μg mRNA encoding EBV gL, and 20 μg mRNA encoding EBV gH.

In still other embodiments, the vaccine comprises 106.7 μg mRNA encoding EBV gp220, 26.7 μg mRNA encoding EBV gp42, 26.7 μg mRNA encoding EBV gL, and 40 μg mRNA encoding EBV gH.

It should be understood that a “total dose” refers to the total amount of mRNA administered, either as a single vaccination or cumulative of an initial dose and any booster dose(s). Thus, if a vaccine is administered as a single dose of 50 μg of mRNA, then the total dose is 50 μg of mRNA. If a vaccine is administered as an initial dose of 50 μg of mRNA then later as a booster dose of 50 μg of mRNA, then the total dose is 100 μg of mRNA.

In some embodiments, a vaccine may be administered intramuscularly, intranasally or intradermally, similarly to the administration of inactivated vaccines known in the art. In preferred embodiments, the vaccine is administered intramuscularly.

The RNA described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

### Dosing Schedule

Prophylactic protection from EBV infection can be achieved following administration of an mRNA vaccine of the present disclosure. Vaccines can be administered once, twice, three times, four times or more but it is likely sufficient to administer the vaccine once (optionally followed by a single booster). It is also possible to administer a vaccine to an infected individual to achieve a therapeutic response. Dosing may need to be adjusted accordingly.

A booster dose refers to an extra administration of the vaccine. A booster (or booster dose or booster vaccine) may be given after an earlier administration of the vaccine. Any two doses of an mRNA vaccine (e.g., an initial dose and a booster dose, or a booster dose and a second booster dose) may be administered, for example, at least one month (˜28, 29, 30, or 31 days) apart, or at least 2, 3, 4, 5, 6, 7, 8, 9, 10, 11, or 12 months apart. In some embodiments, a booster dose is administered yearly.

In some embodiments, a booster dose (e.g., a first, second or third booster dose) is administered at least one month, at least two months, at least three months, at least four months, at least five months, or at least six months after the initial dose or after another booster dose. In some embodiments, a booster dose is administered one month, two months, three months, four months, five months, or six months after the initial dose or after another booster dose. In some embodiments, a second booster dose is administered at least one month, at least two months, at least three months, at least four months, at least five months, or at least six months after a first booster dose. In some embodiments, a second booster dose is administered one month, two months, three months, four months, five months, or six months after a first booster dose. In some embodiments, a booster dose is administered one year or at least one year after the initial dose. In some embodiments, a second booster dose is administered one year or at least one year after a first booster dose.

A method of eliciting an immune response in a subject against an EBV antigen (or multiple antigens) is provided in aspects of the present disclosure. In some embodiments, the method involves administering to the subject a vaccine comprising a mRNA having an open reading frame encoding an EBV antigen, thereby inducing in the subject an immune response specific to the EBV antigen, wherein anti-antigen antibody titer in the subject is increased following vaccination relative to anti-antigen antibody titer in a subject vaccinated with a prophylactically effective dose of a traditional vaccine against the antigen (e.g., EBV antigen).

An anti-antigen antibody is a serum antibody the binds specifically to the antigen (e.g., EBV antigen). In some embodiments, the anti-antigen antibody titer in the subject is increased 1 log to 10 log following vaccination relative to anti-antigen antibody titer in a subject vaccinated with a prophylactically effective dose of a traditional vaccine against the EBV or an unvaccinated subject. In some embodiments, the anti-antigen antibody titer in the subject is increased 1 log, 2 log, 3 log, 4 log. 5 log, or 10 log following vaccination relative to anti-antigen antibody titer in a subject vaccinated with a prophylactically effective dose of a traditional vaccine against the EBV or an unvaccinated subject.

A traditional vaccine, as used herein, refers to a vaccine other than the mRNA vaccines of the present disclosure. For instance, a traditional vaccine includes, but is not limited, to live microorganism vaccines, killed microorganism vaccines, subunit vaccines, protein antigen vaccines, DNA vaccines, virus like particle (VLP) vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

In some embodiments, the immune response in the subject is equivalent to an immune response in a subject vaccinated with a traditional vaccine against the EBV at 2 times to 100 times the dosage level relative to the vaccine. In some embodiments, the immune response in the subject is equivalent to an immune response in a subject vaccinated with a traditional vaccine at twice the dosage level relative to a vaccine of the present disclosure. In some embodiments, the immune response in the subject is equivalent to an immune response in a subject vaccinated with a traditional vaccine at three times the dosage level relative to a vaccine of the present disclosure. In some embodiments, the immune response in the subject is equivalent to an immune response in a subject vaccinated with a traditional vaccine at 4 times, 5 times, 10 times, 50 times, or 100 times the dosage level relative to a vaccine of the present disclosure. In some embodiments, the immune response in the subject is equivalent to an immune response in a subject vaccinated with a traditional vaccine at 10 times to 1000 times the dosage level relative to a vaccine of the present disclosure. In some embodiments, the immune response in the subject is equivalent to an immune response in a subject vaccinated with a traditional vaccine at 100 times to 1000 times the dosage level relative to a vaccine of the present disclosure.

In some embodiments, the immune response in the subject is induced 2 days to 10 weeks earlier relative to an immune response induced in a subject vaccinated with a prophylactically effective dose of a traditional vaccine against EBV. In some embodiments, the immune response in the subject is induced 2 days, 3 days, 1 week, 2 weeks, 3 weeks, 5 weeks, or 10 weeks earlier relative to an immune response induced in a subject vaccinated with a prophylactically effective dose of a traditional vaccine.

In some embodiments, the immune response is assessed by determining [protein] antibody titer in the subject. In other embodiments, the ability of serum or antibody from an immunized subject is tested for its ability to neutralize viral uptake or reduce EBV transformation of human B lymphocytes. In other embodiments, the ability to promote a robust T cell response(s) is measured using art recognized techniques.

### Vaccine Efficacy

Some aspects of the present disclosure provide formulations of the mRNA vaccines, wherein the mRNA is formulated in an effective amount to produce an antigen specific immune response in a subject (e.g., production of antibodies specific to an EBV antigen). “An effective amount” is a dose of the mRNA effective to produce an antigen-specific immune response. Also provided herein are methods of inducing an antigen-specific immune response in a subject.

As used herein, an immune response to a vaccine or LNP of the present disclosure is the development in a subject of a humoral and/or a cellular immune response to a (one or more) EBV protein(s) present in the vaccine. For purposes of the present disclosure, a “humoral” immune response refers to an immune response mediated by antibody molecules, including, e.g., secretory (IgA) or IgG molecules, while a “cellular” immune response is one mediated by T-lymphocytes (e.g., CD4+ helper and/or CD8+ T cells (e.g., CTLs) and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves and antigen-specific response by helper T-cells. Helper T-cells act to help stimulate the function and focus the activity nonspecific effector cells against cells displaying peptide antigens in association with MHC molecules on their surface. A cellular immune response also leads to the production of cytokines, chemokines, and other such molecules produced by activated T-cells and/or other white blood cells including those derived from CD4+ and CD8+ T-cells.

In some embodiments, the antigen-specific immune response is characterized by measuring an anti-EBV antigen antibody titer produced in a subject administered a vaccine as provided herein. An antibody titer is a measurement of the amount of antibodies within a subject, for example, antibodies that are specific to a particular antigen (e.g., an anti-EBV F glycoprotein) or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by an mRNA vaccine.

In some embodiments, an anti-EBV antigen antibody titer produced in a subject is increased by at least 1 log relative to a control. For example, anti-EBV antigen antibody titer produced in a subject may be increased by at least 1.5, at least 2, at least 2.5, or at least 3 log relative to a control. In some embodiments, the anti-EBV antigen antibody titer produced in the subject is increased by 1, 1.5, 2, 2.5 or 3 log relative to a control. In some embodiments, the anti-EBV antigen antibody titer produced in the subject is increased by 1-3 log relative to a control. For example, the anti-EBV antigen antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, the anti-EBV antigen antibody titer produced in a subject is increased at least 2 times relative to a control. For example, the anti-EBV antigen n antibody titer produced in a subject may be increased at least 3 times, at least 4 times, at least 5 times, at least 6 times, at least 7 times, at least 8 times, at least 9 times, or at least 10 times relative to a control. In some embodiments, the anti-EBV antigen antibody titer produced in the subject is increased 2, 3, 4, 5, 6, 7, 8, 9, or 10 times relative to a control. In some embodiments, the anti-EBV antigen antibody titer produced in a subject is increased 2-10 times relative to a control. For example, the anti-EBV antigen antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

In some embodiments, an antigen-specific immune response is measured as a ratio of geometric mean titer (GMT), referred to as a geometric mean ratio (GMR), of serum neutralizing antibody titers to EBV. A geometric mean titer (GMT) is the average antibody titer for a group of subjects calculated by multiplying all values and taking the nth root of the number, where n is the number of subjects with available data.

A control, in some embodiments, is an anti-EBV antigen antibody titer produced in a subject who has not been administered an mRNA vaccine. In some embodiments, a control is an anti-EBV antigen antibody titer produced in a subject administered a recombinant or purified protein vaccine. Recombinant protein vaccines typically include protein antigens that either have been produced in a heterologous expression system (e.g., bacteria or yeast) or purified from large amounts of the pathogenic organism.

In some embodiments, the ability of an mRNA vaccine to be effective is measured in a murine model. For example, a vaccine may be administered to a murine model and the murine model assayed for induction of neutralizing antibody titers. Viral challenge studies may also be used to assess the efficacy of a vaccine of the present disclosure. For example, a vaccine may be administered to a murine model, the murine model challenged with virus, and the murine model assayed for survival and/or immune response (e.g., neutralizing antibody response, T cell response (e.g., cytokine response)).

In some embodiments, an effective amount of an mRNA vaccine is a dose that is reduced compared to the standard of care dose of a recombinant protein vaccine. A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as provided herein, refers to the dose of a recombinant or purified protein vaccine, or a live attenuated or inactivated vaccine, or a VLP vaccine, that a physician/clinician or other medical professional would administer to a subject to treat or prevent EBV infection or a related condition, while following the standard of care guideline for treating or preventing EBV infection or a related condition.

In some embodiments, the anti-EBV antigen antibody titer produced in a subject administered an effective amount of a vaccine is equivalent to an anti-EBV antigen antibody titer produced in a control subject administered a standard of care dose of a recombinant or purified protein vaccine, or a live attenuated or inactivated vaccine, or a VLP vaccine.

Vaccine efficacy may be assessed using standard analyses (sec, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

Efficacy=(ARU−ARV)/ARU×100; and

Efficacy=(1−RR)×100.

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

Effectiveness=(1−OR)×100.

In some embodiments, efficacy of the vaccine is at least 60% relative to unvaccinated control subjects. For example, efficacy of the vaccine may be at least 65%, at least 70%, at least 75%, at least 80%, at least 85%, at least 95%, at least 98%, or 100% relative to unvaccinated control subjects.

Sterilizing Immunity. Sterilizing immunity refers to a unique immune status that prevents effective pathogen infection into the host. In some embodiments, the effective amount of a vaccine of the present disclosure is sufficient to provide sterilizing immunity in the subject for at least 1 year. For example, the effective amount of a vaccine of the present disclosure is sufficient to provide sterilizing immunity in the subject for at least 2 years, at least 3 years, at least 4 years, or at least 5 years. In some embodiments, the effective amount of a vaccine of the present disclosure is sufficient to provide sterilizing immunity in the subject at an at least 5-fold lower dose relative to control. For example, the effective amount may be sufficient to provide sterilizing immunity in the subject at an at least 10-fold lower, 15-fold, or 20-fold lower dose relative to a control.

Detectable Antigen. In some embodiments, the effective amount of a vaccine of the present disclosure is sufficient to produce detectable levels of EBV antigen as measured in serum of the subject at 1-72 hours post administration.

Titer. An antibody titer is a measurement of the amount of antibodies within a subject, for example, antibodies that are specific to a particular antigen (e.g., an anti-EBV antigen). Antibody titer is typically expressed as the inverse of the greatest dilution that provides a positive result. Enzyme-linked immunosorbent assay (ELISA) is a common assay for determining antibody titers, for example.

In some embodiments, the effective amount of a vaccine of the present disclosure is sufficient to produce a 1,000-10,000 neutralizing antibody titer produced by neutralizing antibody against the EBV antigen as measured in serum of the subject at 1-72 hours post administration. In some embodiments, the effective amount is sufficient to produce a 1,000-5,000 neutralizing antibody titer produced by neutralizing antibody against the EBV antigen as measured in serum of the subject at 1-72 hours post administration. In some embodiments, the effective amount is sufficient to produce a 5,000-10,000 neutralizing antibody titer produced by neutralizing antibody against the EBV antigen as measured in serum of the subject at 1-72 hours post administration.

In some embodiments, the neutralizing antibody titer is at least 100 NT₅₀. For example, the neutralizing antibody titer may be at least 200, 300, 400, 500, 600, 700, 800, 900 or 1000 NT₅₀. In some embodiments, the neutralizing antibody titer is at least 10,000 NT₅₀.

In some embodiments, the neutralizing antibody titer is at least 100 neutralizing units per milliliter (NU/mL). For example, the neutralizing antibody titer may be at least 200, 300, 400, 500, 600, 700, 800, 900 or 1000 NU/mL. In some embodiments, the neutralizing antibody titer is at least 10,000 NU/mL.

In some embodiments, an anti-EBV antigen antibody titer produced in the subject is increased by at least 1 log relative to a control. For example, an anti-EBV antigen antibody titer produced in the subject may be increased by at least 2, 3, 4, 5, 6, 7, 8, 9 or 10 log relative to a control.

In some embodiments, an anti-EBV antigen antibody titer produced in the subject is increased at least 2 times relative to a control. For example, an anti-EBV antigen antibody titer produced in the subject is increased by at least 3, 4, 5, 6, 7, 8, 9 or 10 times relative to a control.

In some embodiments, a geometric mean, which is the nth root of the product of n numbers, is generally used to describe proportional growth. Geometric mean, in some embodiments, is used to characterize antibody titer produced in a subject.

A control may be, for example, an unvaccinated subject, or a subject administered a live attenuated viral vaccine, an inactivated viral vaccine, or a protein subunit vaccine.

### EXAMPLES

### Example 1

In vivo studies in Balb/c mice were conducted to evaluate vaccines comprising mRNA encoding soluble EBV gp42 in combination with other EBV antigens. The experimental design is shown in Tables 1 and Table 2. The sequences of the mRNAs used are provided in Table 3, 5

TABLE 1
Studies with mRNA encoding WT EBV gp42

| Group # | Combination | mRNA | μg |
|---|---|---|---|
| 1 | WT gp42 | gH | 0.5 |
| | ⅓ Dose | gL | 0.33 |
| | | gp220 | 1.33 |
| | | wt gp42 | 0.33 |
| 2 | WT gp42 | gH | 1.5 |
| | Dose | gL | 1 |
| | | Gp220 | 4 |
| | | wt gp42 | 1 |
| 3 | WT gp42 | gH | 0.5 |
| | ⅓ Dose + gB | gL | 0.33 |
| | | gp220 | 1.33 |
| | | wt gp42 | 0.33 |
| | | gB | 0.5 |
| 4 | WT gp42 | gH | 1.5 |
| | Dose + gB | gL | 1 |
| | | gp220 | 4 |
| | | wt gp42 | 1 |
| | | gB | 1.5 |

TABLE 2
Studies with mRNA encoding soluble EBV gp42

| Group # | Combination | mRNA | Dose (μg) Ratios |
|---|---|---|---|
| 1 | Sol gp42 | gH | 0.5 |
| | ⅓ Dose | gL | 0.33 |
| | | gp220 | 1.33 |
| | | wt gp42 | 0.33 |
| 2 | Sol gp42 | gH | 1.5 |
| | Dose | gL | 1 |
| | | gp220 | 4 |
| | | wt gp42 | 1 |
| 3 | Sol gp42 | gH | 0.5 |
| | ⅓ Dose + gB | gL | 0.33 |
| | | gp220 | 1.33 |
| | | wt gp42 | 0.33 |
| | | gB | 0.5 |
| 4 | Sol gp42 | gH | 1.5 |
| | Dose + gB | gL | 1 |
| | | gp220 | 4 |
| | | wt gp42 | 1 |
| | | gB | 1.5 |

Two doses of each antigen combination were tested: ‘Dose’ and ‘⅓ Dose’. For each EBV gp42 design, the mRNA encoding EBV gp42 (ORF SEQ ID NO: 10 or 14) was included at equal mass to the mRNA encoding EBV gL (ORF SEQ ID NO: 8). Each gp42 design was tested in the presence and absence of mRNA encoding EBV gB (ORF SEQ ID NO: 12) for each dose. mRNA encoding EBV gH (ORF SEQ ID NO: 6) and mRNA encoding EBV gB (where applicable) were included at 1.5× the mass of mRNA encoding gL and mRNA encoding gp42 to account for stability. The mRNA encoding gp220 (ORF SEQ ID NO: 4) was included at 4× the mass of gL and gp42.

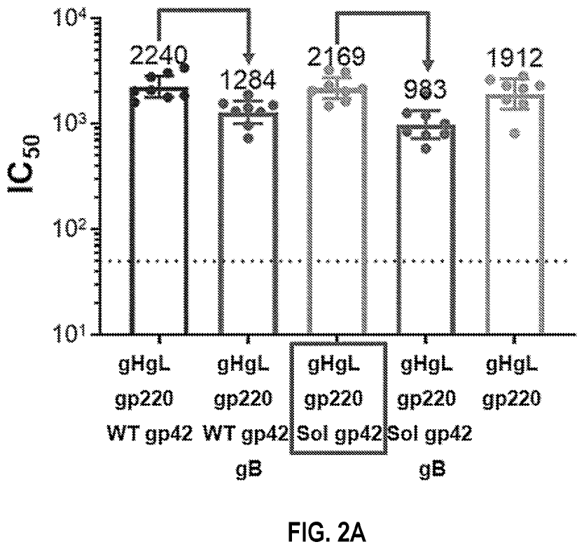

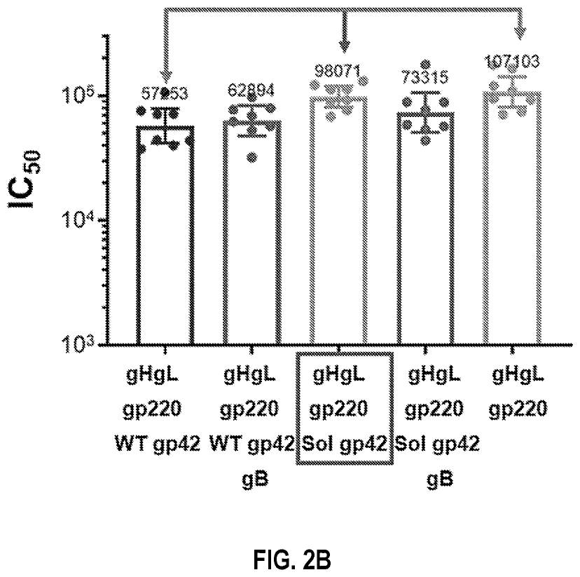

Balb/c mice were vaccinated intramuscularly with the EBV vaccines comprising mRNA encoding the various EBV antigens as set out above in Tables 1 and 2, formulated in a lipid nanoparticle. Neutralization against B cell infection (FIG. 2A) and neutralization against epithelial cell infection (FIG. 2B) was assessed for the ‘Dose’ groups.

These in vivo results show mRNA encoding soluble EBV gp42 adds positively to neutralizing antibody titers produced against B cell infection while not dampening neutralizing antibody titers produced against epithelial cell infection (unlike WT gp42). The addition of mRNA encoding EBV gB was found to cause a drop in neutralizing antibody titers produced against B cell infection when added to vaccines comprising individual mRNAs encoding EBV gH, EBV gL, EBV gp220, and soluble EBV gp42 (similar to WT gp42).

### Example 2

Naïve Balb/c mice were given two doses of a vaccine comprising mRNAs encoding the EBV gp220, sol EBV gp42, EBV gL, and EBV gH proteins of Example 1 approximately three weeks apart. Neutralizing antibody titers against B cell or epithelial cell infection were measured two weeks after the second dose using GFP-labeled virus. Neutralizing antibodies in a set of eight convalescent human sera were measured for comparison.

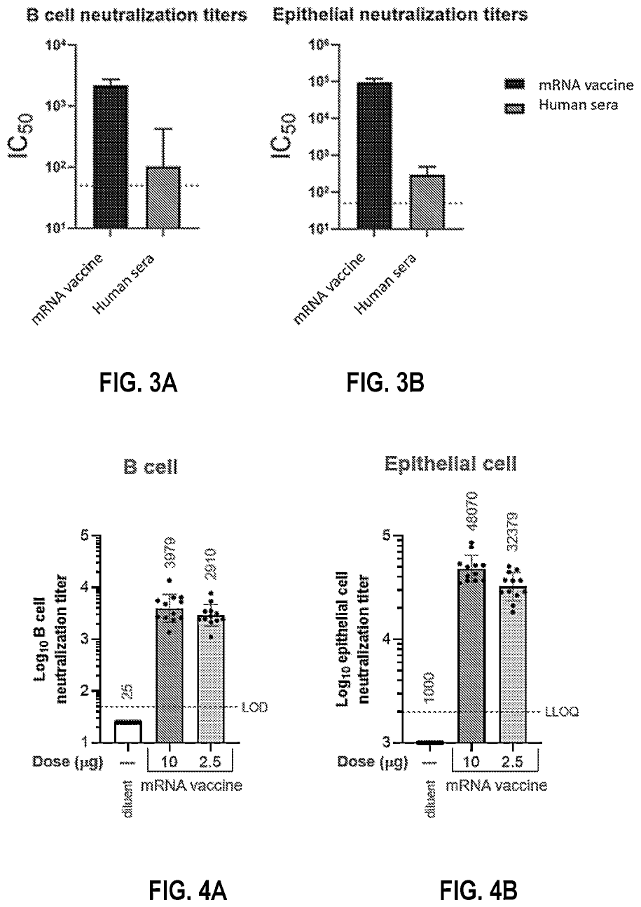

Results shown in FIG. 3 represent eight animals per group and demonstrate high levels of neutralizing antibodies against B cell and epithelial cell infection, and at levels significantly higher than those observed in naturally infected human sera.

### Example 3

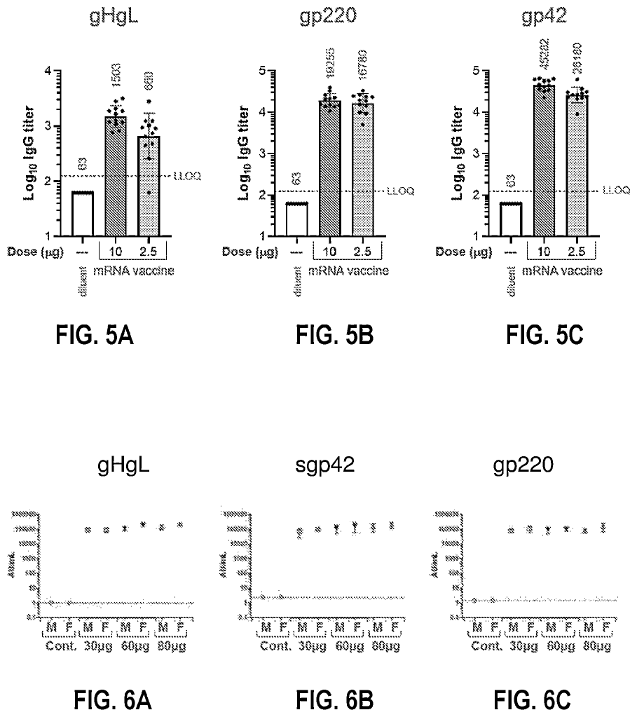

Balb/c mice were immunized twice (3 weeks apart) intramuscularly with diluent or mRNA vaccine encoding gp220, gH, gL and sgp42 (10 μg or 2.5 μg). Sera were collected 2 weeks post the second dose and analyzed for neutralizing and binding antibody levels. Neutralizing antibodies (nAb) were detected in the serum of mice immunized with either dose of mRNA vaccine, but not in mice injected with diluent. Antibodies raised by mRNA vaccine immunization were able to inhibit B cell (FIG. 4A) and epithelial cell infection (FIG. 4B). B cell and epithelial cell nAb titers exhibited a slight (<2-fold) dose-dependent effect. The levels of antigen-specific antibodies in serum were measured using ELISA. Three individual ELISAs were used to measure anti-gHgL (FIG. 5A), anti-gp220 (FIG. 5B) and anti-gp42 (FIG. 5C) IgG. Binding antibodies to each antigen were detected in serum from mice immunized with mRNA vaccine, but not diluent. As was seen with the nAb titers, the overall dose-dependent effect was modest: less than 2-fold for gp220 and gp42, and ˜2.3-fold for gHgL.

### Example 4

Sprague Dawley rats were immunized twice with intramuscular injections of mRNA vaccine at 30 μg. 60 μg or 80 μg, or PBS (control) at a 3 weeks' interval. Antibody titers were measured in serum 2 weeks after the second immunization using a multiplex assay. All animals receiving mRNA vaccine showed detectable antibody titers to gHgL (FIG. 6A), sgp42 (FIG. 6B) and gp220 (FIG. 6C).

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including.” “carrying.” “having.” “containing,” “involving.” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

The entire contents of International Application Nos. PCT/US2015/027400, PCT/US2016/043348, PCT/US2016/058327, PCT/US2016/058324, PCT/US2016/058314, PCT/US2016/058310, and PCT/US2016/058321, are incorporated herein by reference.

### SEQUENCES

It should be understood that any of the mRNA sequences described herein may include a 5′ UTR and/or a 3′ UTR. The UTR sequences may be selected from the following sequences, or other known UTR sequences may be used. It should also be understood that any of the mRNA constructs described herein may further comprise a polyA tail and/or cap (e.g., 7mG(5′)ppp(5′)NlmpNp). Further, while many of the mRNAs and encoded antigen sequences described herein include a signal peptide and/or a peptide tag (e.g., C-terminal His tag), it should be understood that the indicated signal peptide and/or peptide tag may be substituted for a different signal peptide and/or peptide tag, or the signal peptide and/or peptide tag may be omitted.

5′ UTR:
(SEQ ID NO: 24)
GGGAAAUAAGAGAGAAAAGAAGAGUAAGAAGAAAUAUAAGAGCCACC
5′ UTR:
(SEQ ID NO: 1)
GGGAAAUAAGAGAGAAAAGAAGAGUAAGAAGAAAUAUAAGACCCCGGCGC
CGCCACC
3′ UTR:
(SEQ ID NO: 25)
UGAUAAUAGGCUGGAGCCUCGGUGGCCAUGCUUCUUGCCCCUUGGGCCUC
CCCCCAGCCCCUCCUCCCCUUCCUGCACCCGUACCCCCGUGGUCUUUGAA
UAAAGUCUGAGUGGGCGGC
3′ UTR:
(SEQ ID NO: 3)
UGAUAAUAGGCUGGAGCCUCGGUGGCCUAGCUUCUUGCCCCUUGGGCCUC
CCCCCAGCCCCUCCUCCCCUUCCUGCACCCGUACCCCCGUGGUCUUUGAA
UAAAGUCUGAGUGGGCGGC

TABLE 3
Exemplary EBV Sequences
Sequences used in Examples 1 and 2 above

| gp220 | | SEQ ID NO: |
|---|---|---|
| SEQ ID NO: 17 consists of from 5′ end to 3′ end, 5′ UTR SEQ ID NO: 1, mRNA ORF | | 17 |
| SEQ ID NO: 2, and 3′ UTR SEQ ID NO: 3. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGACCCCGGCGCCGCCACC | |
| ORF of mRNA | AUGGAGGCAGCACUGCUGGUCUGCCAGUAUACAAUCCAG | 2 |
| Construct | UCGCUCAUUCAUCUGACAGGCGAGGACCCCGGCUUCUUU | |
| (excluding the stop | AACGUGGAAAUCCCAGAGUUCCCUUUCUAUCCAACCUGU | |
| codon) | AACGUUUGCACUGCUGACGUGAACGUGACAAUCAAUUUC | |
| | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUACAAACCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAACUAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | UCUGUCAAGACCGAGAUGCUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCACUUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUACUGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCACGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCACGCCUCCCCAGAACGCAACCAGUCCUCA | |
| | GGCGCCCAGCGGGCAGAAGACUGCGGUGCCAACUGUGAC | |
| | CAGCACCGGUGGCAAGGCCAACUCAACAACUGGAGGCAA | |
| | GCAUACGACGGGGCACGGCGCCCGGACCUCCACUGAACCC | |
| | ACGACCGAUUACGGAGGUGACAGCACAACACCGCGGCCA | |
| | CGAUAUAAUGCCACCACUUAUCUGCCACCAUCCACAAGCU | |
| | CCAAGCUGCGGCCACGGUGGACCUUCACAAGCCCACCCGU | |
| | GACGACUGCCCAAGCGACGGUGCCAGUGCCACCUACAAGC | |
| | CAGCCACGCUUCUCCAACCUUAGUAUGCUCGUUCUCCAGU | |
| | GGGCCAGCCUUGCUGUUCUGACCCUCCUCCUGCUGCUCGU | |
| | GAUGGCCGAUUGCGCCUUUAGGAGAAACCUCAGCACUAG | |
| | CCACACGUACACCACGCCGCCCUACGAUGACGCCGAGACU | |
| | UACGUC | |
| 3′ UTR | UGAUAAUAGGCUGGAGCCUCGGUGGCCUAGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | MEAALLVCQYTIQSLIHLTGEDPGFFNVEIPEFPFYPTCNVCTA | 4 |
| acid sequence | DVNVTINFDVGGKKHQLDLDFGQLTPHTKAVYQPRGAFGGS | |
| | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNATTTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESTPPQNATSPQAPSGQKT | |
| | AVPTVTSTGGKANSTTGGKHTTGHGARTSTEPTTDYGGDSTT | |
| | PRPRYNATTYLPPSTSSKLRPRWTFTSPPVTTAQATVPVPPTSQ | |
| | PRFSNLSMLVLQWASLAVLTLLLLLVMADCAFRRNLSTSHTY | |
| | TTPPYDDAETYV | |
| PolyA tail | 100 nt | |
| gH | | SEQ ID NO: |
| SEQ ID NO: 18 consists of from 5′ end to 3′ end, 5′ UTR SEQ ID NO: 1, mRNA ORF | | 18 |
| SEQ ID NO: 5, and 3′ UTR SEQ ID NO: 3. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGACCCCGGCGCCGCCACC | |
| ORF of mRNA | AUGCAGUUGUUGUGCGUGUUCUGCCUCGUGUUACUCUGG | 5 |
| Construct | GAGGUGGGCGCCGCCAGCCUUAGCGAGGUGAAGCUCCAC | |
| (excluding the stop | UUGGACAUCGAGGGCCACGCCAGCCACUACACCAUCCCCU | |
| codon) | GGACCGAGCUCAUGGCCAAGGUGCCCGGCCUUAGCCCCGA | |
| | GGCCCUGUGGCGGGAGGCCAACGUGACCGAGGACCUGGC | |
| | CAGCAUGCUGAACCGGUACAAGCUGAUCUACAAGACCAG | |
| | CGGCACCCUGGGCAUCGCCCUGGCCGAGCCCGUGGACAUC | |
| | CCCGCCGUUAGCGAAGGCAGCAUGCAGGUGGACGCCAGC | |
| | AAGGUGCACCCCGGCGUGAUCAGCGGCCUGAACAGCCCCG | |
| | CCUGUAUGUUGAGCGCCCCACUGGAGAAGCAGCUGUUCU | |
| | ACUACAUCGGCACCAUGCUGCCCAACACCCGGCCCCACAG | |
| | CUACGUGUUCUACCAGCUGCGGUGCCACCUGAGCUACGU | |
| | UGCCCUGAGCAUCAACGGCGACAAGUUCCAGUACACCGG | |
| | CGCCAUGACCAGCAAGUUCCUGAUGGGCACCUACAAGCG | |
| | GGUCACCGAGAAGGGCGACGAGCACGUGCUGUCACUGGU | |
| | GUUCGGCAAGACCAAGGACCUGCCCGACCUGCGGGGCCCC | |
| | UUCAGCUACCCUAGUUUGACCAGCGCCCAGAGCGGCGAC | |
| | UACAGCUUGGUGAUCGUGACCACCUUCGUGCACUACGCC | |
| | AACUUCCACAACUACUUCGUGCCCAACCUGAAGGACAUG | |
| | UUCAGCCGGGCCGUGACCAUGACUGCCGCUUCUUACGCCC | |
| | GGUACGUGCUGCAGAAGCUGGUCCUGCUGGAGAUGAAGG | |
| | GCGGCUGCCGGGAGCCCGAGCUGGACACCGAAACACUGA | |
| | CCACCAUGUUCGAGGUGAGCGUGGCCUUCUUCAAGGUGG | |
| | GUCACGCGGUGGGCGAAACCGGCAACGGCUGCGUGGACU | |
| | UACGCUGGCUGGCCAAGAGCUUCUUCGAGCUGACCGUGC | |
| | UGAAGGAUAUCAUCGGCAUCUGCUACGGCGCCACCGUGA | |
| | AGGGCAUGCAGAGCUACGGCCUGGAGCGGCUGGCCGCCA | |
| | UGCUUAUGGCAACAGUGAAGAUGGAGGAGCUGGGACACC | |
| | UGACAACAGAGAAGCAGGAGUACGCCCUGAGACUGGCCA | |
| | CAGUGGGCUACCCAAAGGCCGGCGUGUACAGUGGACUGA | |
| | UCGGCGGCGCAACCAGCGUGCUGCUAUCCGCUUACAACCG | |
| | GCACCCGCUGUUCCAGCCCCUGCACACCGUGAUGCGGGAA | |
| | ACCCUGUUCAUCGGAAGCCACGUCGUGCUGCGGGAGCUG | |
| | AGGCUGAACGUAACCACCCAGGGCCCUAAUCUGGCCCUG | |
| | UAUCAGCUCCUCAGUACCGCCCUGUGCAGCGCCCUUGAGA | |
| | UCGGCGAGGUGCUCAGAGGCCUGGCCCUCGGUACCGAGA | |
| | GCGGCCUCUUCAGCCCAUGCUACUUAAGCCUGCGGUUCG | |
| | ACCUGACCCGGGACAAGUUGCUGAGCAUGGCCCCGCAGG | |
| | AGGCCACACUGGACCAGGCAGCUGUAUCCAACGCCGUGG | |
| | ACGGCUUCCUGGGCAGACUGUCCCUGGAACGGGAGGACC | |
| | GGGACGCCUGGCACCUGCCUGCCUACAAGUGUGUGGAUC | |
| | GGCUGGACAAGGUGCUGAUGAUCAUCCCUCUGAUUAAUG | |
| | UCACCUUCAUCAUCAGCAGCGACCGGGAGGUGCGGGGAU | |
| | CCGCCCUCUACGAGGCCAGCACCACCUAUCUGAGCAGCAG | |
| | CCUGUUCCUGUCUCCUGUGAUCAUGAACAAGUGCAGCCA | |
| | GGGCGCCGUGGCCGGCGAGCCCCGGCAGAUCCCCAAGAUC | |
| | CAGAACUUCACCCGGACCCAGAAGUCUUGCAUCUUCUGC | |
| | GGCUUCGCCCUUUUGUCCUACGACGAGAAGGAGGGCUUG | |
| | GAGACUACAACCUACAUCACCAGCCAGGAGGUGCAGAAC | |
| | AGCAUCCUGUCAUCUAAUUACUUCGACUUCGACAACCUG | |
| | CACGUUCAUUACCUGCUCCUCACCACCAACGGUACCGUCA | |
| | UGGAAAUCGCCGGACUGUACGAGGAGCGGGCCCAUGUUG | |
| | UGCUGGCCAUCAUCCUGUACUUCAUCGCUUUCGCACUUG | |
| | GCAUCUUCCUGGUGCACAAGAUCGUGAUGUUCUUCCUG | |
| 3′ UTR | UGAUAAUAGGCUGGAGCCUCGGUGGCCUAGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | MQLLCVFCLVLLWEVGAASLSEVKLHLDIEGHASHYTIPWTE | 6 |
| acid sequence | LMAKVPGLSPEALWREANVTEDLASMLNRYKLIYKTSGTLGI | |
| | ALAEPVDIPAVSEGSMQVDASKVHPGVISGLNSPACMLSAPLE | |
| | KQLFYYIGTMLPNTRPHSYVFYQLRCHLSYVALSINGDKFQYT | |
| | GAMTSKFLMGTYKRVTEKGDEHVLSLVFGKTKDLPDLRGPFS | |
| | YPSLTSAQSGDYSLVIVTTFVHYANFHNYFVPNLKDMFSRAV | |
| | TMTAASYARYVLQKLVLLEMKGGCREPELDTETLTTMFEVS | |
| | VAFFKVGHAVGETGNGCVDLRWLAKSFFELTVLKDIIGICYG | |
| | ATVKGMQSYGLERLAAMLMATVKMEELGHLTTEKQEYALR | |
| | LATVGYPKAGVYSGLIGGATSVLLSAYNRHPLFQPLHTVMRE | |
| | TLFIGSHVVLRELRLNVTTQGPNLALYQLLSTALCSALEIGEVL | |
| | RGLALGTESGLFSPCYLSLRFDLTRDKLLSMAPQEATLDQAA | |
| | VSNAVDGFLGRLSLEREDRDAWHLPAYKCVDRLDKVLMIIPL | |
| | INVTFIISSDREVRGSALYEASTTYLSSSLFLSPVIMNKCSQGAV | |
| | AGEPRQIPKIQNFTRTQKSCIFCGFALLSYDEKEGLETTTYITSQ | |
| | EVQNSILSSNYFDFDNLHVHYLLLTTNGTVMEIAGLYEERAH | |
| | VVLAIILYFIAFALGIFLVHKIVMFFL | |
| PolyA tail | 100 nt | |
| gL | | SEQ ID NO: |
| SEQ ID NO: 19 consists of from 5′ end to 3′ end, 5′ UTR SEQ ID NO: 1, mRNA ORF | | 19 |
| SEQ ID NO: 7, and 3′ UTR SEQ ID NO: 3. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGACCCCGGCGCCGCCACC | |
| ORF of mRNA | AUGAGAGCCGUGGGCGUGUUCCUGGCCAUCUGCCUGGUG | 7 |
| Construct | ACCAUCUUCGUGCUGCCCACCUGGGGCAACUGGGCCUACC | |
| (excluding the stop | CCUGCUGCCACGUGACCCAGCUGAGAGCCCAGCACCUGCU | |
| codon) | GGCCCUGGAGAACAUCAGCGACAUCUACCUGGUGAGCAA | |
| | CCAGACCUGCGACGGCUUCAGCCUGGCCAGCCUGAACAGC | |
| | CCCAAGAACGGCAGCAACCAGCUGGUGAUCAGCAGAUGC | |
| | GCCAACGGCCUGAACGUGGUGAGCUUCUUCAUCAGCAUC | |
| | CUGAAGAGAAGCAGCAGCGCCCUGACCGGCCACCUGAGA | |
| | GAGCUGCUGACCACCCUGGAGACCCUGUACGGCAGCUUC | |
| | AGCGUGGAGGACCUGUUCGGCGCCAACCUGAACAGAUAC | |
| | GCCUGGCACAGAGGCGGC | |
| 3′ UTR | UGAUAAUAGGCUGGAGCCUCGGUGGCCUAGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | MRAVGVFLAICLVTIFVLPTWGNWAYPCCHVTQLRAQHLLA | 8 |
| acid sequence | LENISDIYLVSNQTCDGFSLASLNSPKNGSNQLVISRCANGLNV | |
| | VSFFISILKRSSSALTGHLRELLTTLETLYGSFSVEDLFGANLNR | |
| | YAWHRGG | |
| PolyA tail | 100 nt | |
| sol gp42 | | SEQ ID NO: |
| SEQ ID NO: 20 consists of from 5′ end to 3′ end, 5′ UTR SEQ ID NO: 1, mRNA ORF | | 20 |
| SEQ ID NO: 9, and 3′ UTR SEQ ID NO: 3. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGACCCCGGCGCCGCCACC | |
| ORF of mRNA | AUGACCCGGCGGCGGGUGUUAAGCGUGGUGGUGCUCUUA | 9 |
| Construct | GCCGCCUUAGCCUGCCGGCUUGGCGCCAUCACCUGGGUGC | |
| (excluding the stop | CCAAGCCCAACGUGGAGGUGUGGCCCGUGGACCCGCCACC | |
| codon) | UCCCGUGAACUUCAACAAGACCGCCGAGCAGGAGUACGG | |
| | CGACAAGGAGGUGAAGCUGCCCCACUGGACGCCGACCCU | |
| | GCACACCUUCCAGGUGCCCCAGAACUACACCAAGGCCAAC | |
| | UGCACCUACUGCAACACCAGAGAGUACACCUUCAGCUAC | |
| | AAGGGCUGCUGCUUCUACUUCACCAAGAAGAAGCACACC | |
| | UGGAACGGCUGCUUCCAGGCCUGCGCCGAGCUGUACCCCU | |
| | GCACCUACUUCUACGGCCCCACCCCAGACAUCCUGCCCGU | |
| | GGUGACCAGAAACCUGAACGCCAUCGAGAGCCUGUGGGU | |
| | GGGCGUGUACAGAGUGGGCGAGGGCAACUGGACCAGCCU | |
| | GGACGGCGGCACCUUCAAGGUGUACCAGAUCUUCGGCAG | |
| | CCACUGCACCUACGUGAGCAAGUUCAGCACCGUGCCCGUG | |
| | AGCCACCACGAGUGCAGCUUCCUGAAGCCCUGCCUGUGCG | |
| | UGAGCCAGAGAAGCAACAGC | |
| 3′ UTR | UGAUAAUAGGCUGGAGCCUCGGUGGCCUAGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGGG | |
| | C | |
| Corresponding amino | MTRRRVLSVVVLLAALACRLGAITWVPKPNVEVWPVDPPPPV | 10 |
| acid sequence | NFNKTAEQEYGDKEVKLPHWTPTLHTFQVPQNYTKANCTYC | |
| | NTREYTFSYKGCCFYFTKKKHTWNGCFQACAELYPCTYFYGP | |
| | TPDILPVVTRNLNAIESLWVGVYRVGEGNWTSLDGGTFKVYQ | |
| | IFGSHCTYVSKFSTVPVSHHECSFLKPCLCVSQRSNS | |
| PolyA tail | 100 nt | |
| gB | | SEQ ID NO: |
| SEQ ID NO: 21 consists of from 5′ end to 3′ end, 5′ UTR SEQ ID NO: 1, mRNA ORF | | 21 |
| SEQ ID NO: 11, and 3′ UTR SEQ ID NO: 3. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGACCCCGGCGCCGCCACC | |
| ORF of mRNA | AUGACCCGGCGGCGGGUGUUAAGCGUGGUGGUGCUCUUA | 11 |
| Construct | GCCGCCUUAGCCUGCCGGCUUGGCGCCCAGACUCCGGAGC | |
| (excluding the stop | AGCCCGCUCCUCCCGCCACCACCGUCCAGCCCACCGCCAC | |
| codon) | CCGCCAGCAGACCUCCUUCCCCUUCCGCGUCUGCGAGCUC | |
| | UCCUCCCACGGCGACCUCUUCCGCUUCUCCUCCGACAUCC | |
| | AGUGCCCCUCCUUCGGCACCCGCGAGAACCACACCGAGGG | |
| | CCUCCUCAUGGUCUUCAAGGACAACAUCAUCCCCUACUCC | |
| | UUCAAGGUCCGCUCCUACACCAAGAUCGUCACCAACAUCC | |
| | UCAUCUACAACGGCUGGUACGCCGACUCUGUGACUAAUC | |
| | GCCACGAGGAGAAGUUCUCCGUCGACUCCUACGAGACUG | |
| | ACCAGAUGGACACCAUCUACCAGUGCUACAACGCCGUCA | |
| | AGAUGACCAAGGACGGCCUCACCCGCGUCUACGUCGACCG | |
| | CGACGGCGUCAACAUCACCGUCAACCUCAAGCCUACAGGC | |
| | GGCCUCGCCAACGGCGUCCGCCGCUACGCCUCCCAGACCG | |
| | AGCUCUACGACGCCCCUGGCUGGCUCAUCUGGACCUACCG | |
| | CACCAGGACCACUGUCAAUUGCCUCAUCACCGACAUGAU | |
| | GGCCAAGUCCAACUCCCCUUUCGACUUCUUCGUGACCACC | |
| | ACAGGCCAGACCGUCGAGAUGUCGCCAUUCUAUGACGGC | |
| | AAGAACAAGGAGACUUUCCACGAGAGAGCGGACUCUUUC | |
| | CAUGUCCGCACCAACUACAAGAUUGUCGACUACGACAAC | |
| | CGCGGCACCAAUCCACAGGGCGAGCGCCGCGCCUUCCUCG | |
| | ACAAGGGCACCUACACCCUCUCCUGGAAGCUCGAGAACCG | |
| | CACCGCCUACUGUCCUCUCCAGCACUGGCAGACCUUCGAU | |
| | UCCACCAUCGCCACCGAGACUGGCAAGUCCAUCCAUUUCG | |
| | UCACCGACGAAGGAACAUCUUCAUUCGUCACAAAUACAA | |
| | CCGUGGGCAUCGAGCUCCCCGACGCCUUCAAGUGCAUCGA | |
| | GGAGCAGGUCAACAAGACCAUGCACGAGAAGUACGAGGC | |
| | CGUCCAGGACCGGUAUACAAAGGGCCAGGAGGCCAUCAC | |
| | CUACUUCAUCACCAGCGGAGGUCUGCUUCUCGCCUGGCU | |
| | UCCACUCACACCUCGCUCCCUGGCCACAGUGAAGAACCUC | |
| | ACUGAGUUGACCACCCCAACAAGUUCCCCACCUAGUAGUC | |
| | CUAGCCCUCCUGCUCCUUCCGCCGCCCGCGGCUCCACGCC | |
| | UGCCGCCGUCCUGCGGAGACGGAGAAGGGACGCCGGCAA | |
| | CGCUACCACUCCUGUGCCGCCGACAGCCCCUGGAAAGUCC | |
| | CUAGGUACCCUCAACAACCCAGCGACAGUUCAGAUCCAG | |
| | UUCGCCUACGACUCUCUCAGGCGCCAGAUCAACCGCAUGC | |
| | UUGGUGACCUUGCCCGCGCCUGGUGCCUCGAGCAGAAGC | |
| | GCCAGAACAUGGUGCUUCGUGAACUAACAAAGAUUAAUC | |
| | CUACUACAGUGAUGUCCUCCAUCUACGGCAAGGCCGUCG | |
| | CCGCCAAGCGCCUCGGCGACGUCAUCUCCGUCUCCCAGUG | |
| | CGUCCCAGUGAACCAGGCUACCGUGACCCUCAGGAAGUCC | |
| | AUGAGAGUGCCAGGCUCCGAGACAAUGUGCUACUCCCGC | |
| | CCACUCGUCUCCUUCUCCUUCAUCAACGACACAAAGACCU | |
| | ACGAGGGCCAGUUAGGCACUGACAACGAGAUCUUCUUGA | |
| | CUAAGAAGAUGACUGAGGUAUGCCAGGCAACUUCUCAAU | |
| | ACUACUUCCAGUCUGGAAAUGAGAUCCACGUAUAUAACG | |
| | ACUACCACCACUUCAAGACUAUUGAACUCGACGGAAUUG | |
| | CCACCCUCCAAACAUUCAUCUCACUGAAUACCUCCCUCAU | |
| | CGAGAACAUCGACUUCGCCUCCCUCGAGCUGUAUAGCAG | |
| | AGACGAGCAGCGCGCCUCCAACGUCUUCGACCUCGAGGGC | |
| | AUCUUCCGCGAGUACAACUUCCAGGCACAGAACAUAGCC | |
| | GGCCUCCGUAAGGAUCUGGACAAUGCCGUGUCCAACGGC | |
| | CGCAACCAGUUCGUCGACGGUUUGGGUGAACUCAUGGAC | |
| | UCUCUGGGCUCCGUCGGCCAGUCCAUAACUAACUUAGUC | |
| | UCUACGGUGGGAGGCCUAUUCAGCAGCCUGGUGAGCGGC | |
| | UUCAUCUCUUUCUUCAAGAACCCCUUCGGCGGCAUGCUC | |
| | AUCCUCGUCCUCGUCGCCGGCGUCGUCAUACUGGUGAUC | |
| | UCACUCACAAGGAGGACGCGCCAAAUGUCCCAGCAGCCA | |
| | GUGCAGAUGCUCUACCCAGGCAUAGACGAGCUCGCUCAG | |
| | CAGCACGCGUCGGGUGAGGGACCAGGCAUCAAUCCUAUC | |
| | UCCAAGACUGAGCUGCAAGCCAUUAUGCUCGCCCUCCACG | |
| | AGCAGAACCAGGAACAGAAGCGGGCCGCCCAGCGAGCUG | |
| | CCGGCCCCUCCGUCGCCAGUAGGGCACUACAAGCCGCCCG | |
| | GGACCGCUUCCCAGGCCUGAGAAGGAGGAGAUACCACGA | |
| | CCCAGAGACAGCCGCUGCCCUCCUUGGCGAAGCAGAAACC | |
| | GAGUUC | |
| 3′ UTR | UGAUAAUAGGCUGGAGCCUCGGUGGCCUAGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | MTRRRVLSVVVLLAALACRLGAQTPEQPAPPATTVQPTATRQ | 12 |
| acid sequence | QTSFPFRVCELSSHGDLFRFSSDIQCPSFGTRENHTEGLLMVFK | |
| | DNIIPYSFKVRSYTKIVTNILIYNGWYADSVTNRHEEKFSVDSY | |
| | ETDQMDTIYQCYNAVKMTKDGLTRVYVDRDGVNITVNLKPT | |
| | GGLANGVRRYASQTELYDAPGWLIWTYRTRTTVNCLITDMM | |
| | AKSNSPFDFFVTTTGQTVEMSPFYDGKNKETFHERADSFHVR | |
| | TNYKIVDYDNRGTNPQGERRAFLDKGTYTLSWKLENRTAYC | |
| | PLQHWQTFDSTIATETGKSIHFVTDEGTSSFVTNTTVGIELPDA | |
| | FKCIEEQVNKTMHEKYEAVODRYTKGQEAITYFITSGGLLLA | |
| | WLPLTPRSLATVKNLTELTTPTSSPPSSPSPPAPSAARGSTPAA | |
| | VLRRRRRDAGNATTPVPPTAPGKSLGTLNNPATVQIQFAYDSL | |
| | RRQINRMLGDLARAWCLEQKRQNMVLRELTKINPTTVMSSIY | |
| | GKAVAAKRLGDVISVSQCVPVNQATVTLRKSMRVPGSETMC | |
| | YSRPLVSFSFINDTKTYEGQLGTDNEIFLTKKMTEVCQATSQY | |
| | YFQSGNEIHVYNDYHHFKTIELDGIATLQTFISLNTSLIENIDFA | |
| | SLELYSRDEQRASNVFDLEGIFREYNFQAQNIAGLRKDLDNAV | |
| | SNGRNQFVDGLGELMDSLGSVGQSITNLVSTVGGLFSSLVSGF | |
| | ISFFKNPFGGMLILVLVAGVVILVISLTRRTRQMSQQPVQMLY | |
| | PGIDELAQQHASGEGPGINPISKTELQAIMLALHEQNQEQKRA | |
| | AQRAAGPSVASRALQAARDRFPGLRRRRYHDPETAAALLGE | |
| | AETEF | |
| PolyA tail | 100 nt | |
| WT gp42 | | SEQ ID NO: |
| SEQ ID NO: 22 consists of from 5′ end to 3′ end, 5′ UTR SEQ ID NO: 1, mRNA ORF | | 22 |
| SEQ ID NO: 13, and 3′ UTR SEQ ID NO: 3. | | |
| Chemistry | 1-methylpseudouridine | |
| Cap | 7mG(5′)ppp(5′)NImpNp | |
| 5′ UTR | GGGAAAUAAGAGAGAAAAGAAGAGUAAGAAGAAAUAUA | 1 |
| | AGACCCCGGCGCCGCCACC | |
| ORF of mRNA | AUGGUGAGCUUCAAGCAGGUGAGAGUGCCCCUGUUCACC | 13 |
| Construct | GCCAUCGCCCUGGUGAUCGUGCUGCUGCUGGCCUACUUCC | |
| (excluding the stop | UGCCACCCAGAGUGAGAGGCGGCGGCAGAGUGGCCGCCG | |
| codon) | CCGCCAUCACCUGGGUGCCCAAGCCCAACGUGGAGGUGU | |
| | GGCCCGUGGACCCGCCACCUCCCGUGAACUUCAACAAGAC | |
| | CGCCGAGCAGGAGUACGGCGACAAGGAGGUGAAGCUGCC | |
| | CCACUGGACGCCGACCCUGCACACCUUCCAGGUGCCCCAG | |
| | AACUACACCAAGGCCAACUGCACCUACUGCAACACCAGAG | |
| | AGUACACCUUCAGCUACAAGGGCUGCUGCUUCUACUUCA | |
| | CCAAGAAGAAGCACACCUGGAACGGCUGCUUCCAGGCCU | |
| | GCGCCGAGCUGUACCCCUGCACCUACUUCUACGGCCCCAC | |
| | CCCAGACAUCCUGCCCGUGGUGACCAGAAACCUGAACGCC | |
| | AUCGAGAGCCUGUGGGUGGGCGUGUACAGAGUGGGCGAG | |
| | GGCAACUGGACCAGCCUGGACGGCGGCACCUUCAAGGUG | |
| | UACCAGAUCUUCGGCAGCCACUGCACCUACGUGAGCAAG | |
| | UUCAGCACCGUGCCCGUGAGCCACCACGAGUGCAGCUUCC | |
| | UGAAGCCCUGCCUGUGCGUGAGCCAGAGAAGCAACAGC | |
| 3′ UTR | UGAUAAUAGGCUGGAGCCUCGGUGGCCUAGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | MVSFKQVRVPLFTAIALVIVLLLAYFLPPRVRGGGRVAAAAIT | 14 |
| acid sequence | WVPKPNVEVWPVDPPPPVNFNKTAEQEYGDKEVKLPHWTPT | |
| | LHTFQVPQNYTKANCTYCNTREYTFSYKGCCFYFTKKKHTW | |
| | NGCFQACAELYPCTYFYGPTPDILPVVTRNLNAIESLWVGVYR | |
| | VGEGNWTSLDGGTFKVYQIFGSHCTYVSKFSTVPVSHHECSFL | |
| | KPCLCVSQRSNS | |
| PolyA tail | 100 nt | |
| gp350 | | SEQ ID NO: |
| SEQ ID NO: 23 consists of from 5′ end to 3′ end, 5′ UTR SEQ ID NO: 1, mRNA ORF | | 23 |
| SEQ ID NO: 15, and 3′ UTR SEQ ID NO: 3. | | |
| Chemistry | 1-methylpseudouridine | |
| Cap | 7mG(5′)ppp(5′)NImpNp | |
| 5′ UTR | GGGAAAUAAGAGAGAAAAGAAGAGUAAGAAGAAAUAUA | 1 |
| | AGACCCCGGCGCCGCCACC | |
| ORF of mRNA | AUGGAGGCAGCACUGCUGGUCUGCCAGUAUACAAUCCAG | 15 |
| Construct | UCGCUCAUUCAUCUGACAGGCGAGGACCCCGGCUUCUUU | |
| (excluding the stop | AACGUGGAAAUCCCAGAGUUCCCUUUCUAUCCAACCUGU | |
| codon) | AACGUUUGCACUGCUGACGUGAACGUGACAAUCAAUUUC | |
| | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUACAAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAACUAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | UCUGUCAAGACCGAGAUGCUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCACUUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUACUGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCACGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′ UTR | UGAUAAUAGGCUGGAGCCUCGGUGGCCUAGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | MEAALLVCQYTIQSLIHLTGEDPGFFNVEIPEFPFYPTCNVCTA | 16 |
| acid sequence | DVNVTINFDVGGKKHQLDLDFGQLTPHTKAVYQPRGAFGGS | |
| | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNATTTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

It should be understood that any one of the sequences of Table 3 may be fully modified by N1-methylpseudoruidine.

What is claimed is:

1. A vaccine, comprising a lipid nanoparticle that comprises:
(a) a messenger ribonucleic acid (mRNA) comprising an open reading frame encoding an Epstein-Barr virus (EBV) glycoprotein 220 (gp220);
(b) an mRNA comprising an open reading frame encoding glycoprotein 42 (gp42);
(c) an mRNA comprising an open reading frame encoding glycoprotein L (gL); and
(d) an mRNA comprising an open reading frame encoding glycoprotein H (gH).

2. The vaccine of claim 1, wherein the gp42 is a soluble form of gp42.

3. The vaccine of claim 1 or 2, wherein the mRNA of (a) is at 5×, 4.5×, 4×, 3.5×, 3×, 2.5×, 2×, or 1.5× the mRNA of (b), (c), and/or (d).

4. The vaccine of claim 1 or 2, wherein the mRNA of (a) is at equal mass to the mRNA of (b), (c), and/or (d).

5. The vaccine of claim 1 or 2, wherein the mRNA of (b) is at 5×, 4.5×, 4×, 3.5×, 3×, 2.5×, 2×, or 1.5× the mRNA of (a), (c), and/or (d).

6. The vaccine of claim 1 or 2, wherein the mRNA of (b) is at equal mass to the mRNA of a), (c), and/or (d).

7. The vaccine of claim 1 or 2, wherein the mRNA of (c) is at 5×, 4.5×, 4×, 3.5×, 3×, 2.5×, 2×, or 1.5× the mRNA of (a), (b), and/or (d).

8. The vaccine of claim 1 or 2, wherein the mRNA of (c) is at equal mass to the mRNA of (a), (b), and/or (d).

9. The vaccine of claim 1 or 2, wherein the mRNA of (d) is at 5×, 4.5×, 4×, 3.5×, 3×, 2.5×, 2×, or 1.5× the mRNA of (a), (b), and/or (c).

10. The vaccine of claim 1 or 2, wherein the mRNA of (d) is at equal mass to the mRNA of (a), (b), and/or (c).

11. The vaccine of claim 1 or 2, wherein the mass ratio of (a):(b):(c):(d) is 4:1:1:1.5.

12. The vaccine of claim 1 or 2, wherein the mass ratio of (a):(b):(c):(d) is 4:1:1:1.

13. The vaccine of claim 1 or 2, wherein the vaccine comprises 26.7 μg mRNA encoding EBV gp220, 6.7 μg mRNA encoding EBV gp42, 6.7 μg mRNA encoding EBV gL, and 10 μg mRNA encoding EBV gH.

14. The vaccine of claim 1 or 2, wherein the vaccine comprises 53.3 μg mRNA encoding EBV gp220, 13.3 μg mRNA encoding EBV gp42, 13.3 μg mRNA encoding EBV gL, and 20 μg mRNA encoding EBV gH.

15. The vaccine of any one of the preceding claims, wherein the lipid nanoparticle further comprises (e) an mRNA comprising an open reading frame encoding glycoprotein B.

16. The vaccine of claim 15, wherein the mass ratio of (a):(b):(c):(d):(e) is 4:1:1:1.5:1.5.

17. The vaccine of any one of the preceding claims, wherein the gp220 comprises an amino acid sequence having at least 85%, at least 90%, at least 95%, or at least 98% identity to the amino acid sequence of SEQ ID NO: 4.

18. The vaccine of claim 17, wherein the gp220 comprises the amino acid sequence of SEQ ID NO: 4.

19. The vaccine of any one of the preceding claims, wherein the gL comprises an amino acid sequence having at least 85%, at least 90%, at least 95%, or at least 98% identity to the amino acid sequence of SEQ ID NO: 8.

20. The vaccine of claim 19, wherein the gL comprises the amino acid sequence of SEQ ID NO: 8.

21. The vaccine of any one of the preceding claims, wherein the gH comprises an amino acid sequence having at least 85%, at least 90%, at least 95%, or at least 98% identity to the amino acid sequence of SEQ ID NO: 6.

22. The vaccine of claim 21, wherein the gH comprises the amino acid sequence of SEQ ID NO: 6.

23. The vaccine of any one of the preceding claims, wherein the gp42 comprises an amino acid sequence having at least 85%, at least 90%, at least 95%, or at least 98% identity to the amino acid sequence of SEQ ID NO: 14.

24. The vaccine of claim 23, wherein the gp42 comprises the amino acid sequence of SEQ ID NO: 14.

25. The vaccine of any one of claims 2-24, wherein the soluble gp42 comprises an amino acid sequence having at least 85%, at least 90%, at least 95%, or at least 98% identity to the amino acid sequence of SEQ ID NO: 10.

26. The vaccine of claim 25, wherein the soluble gp42 comprises the amino acid sequence of SEQ ID NO: 10.

27. The vaccine of any one of claims 15-26, wherein the gB comprises an amino acid sequence having at least 85%, at least 90%, at least 95%, or at least 98% identity to the amino acid sequence of SEQ ID NO: 12.

28. The vaccine of claim 27, wherein the gB comprises the amino acid sequence of SEQ ID NO: 12.

29. The vaccine of any one of the preceding claims, wherein the mRNA of any one or more of (a)-(e) comprises a 5′ 7mG(5′)ppp(5′)NlmpNp cap and a 3′ polyA tail.

30. The vaccine of any one of the preceding claims, wherein the mRNA of any one or more of (a)-(e) comprises a 1-methylpseudourine chemical modification.

31. The vaccine of any one of the preceding claims, wherein the lipid nanoparticle comprises 45-55 mol % ionizable amino lipid, 15-20 mol % neutral lipid, 35-45 mol % cholesterol, and 0.5-5 mol % PEG-modified lipid.

32. The vaccine of any one of the preceding claims, wherein the ionizable amino lipid is Compound I:

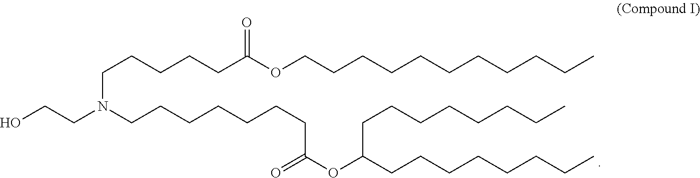 [SMILES: CCCCCCCCCCCOC(=O)CCCCCN(CCO)CCCCCCCC(=O)OC(CCCCCCCC)CCCCCCCC]

33. The vaccine of any one of the preceding claims, wherein the lipid nanoparticle comprises 50 mol % ionizable amino lipid.

34. The vaccine of any one of the preceding claims, wherein the lipid nanoparticle comprises 49 mol % ionizable amino lipid.

35. The vaccine of any one of the preceding claims, wherein the lipid nanoparticle comprises 48 mol % ionizable amino lipid.

36. A method comprising administering to a subject the vaccine of any one of the preceding claims in an amount effective to induce an immune response to EBV.

37. The method of claim 36, wherein the vaccine is administered as a single dose.

38. The method of claim 36, wherein the vaccine is administered as an initial dose and as at least one booster dose.


SEQUENCE LISTING (34 sequences)

SEQ ID NO: 1 - RNA, 57 nt, Artificial Sequence
  Synthetic
gggaaauaagagagaaaagaagaguaagaagaaauauaagaccccggcgccgccacc

SEQ ID NO: 2 - RNA, 1974 nt, Artificial Sequence
  Synthetic
auggaggcagcacugcuggucugccaguauacaauccagucgcucauucaucugacaggcgaggaccccggcuucuuuaacguggaaaucccagaguucccuuucuauccaaccuguaacguuugcacugcugacgugaacgugacaaucaauuucgacgugggaggaaagaagcaucagcuugaucuggauuucggccaacugacgccacacaccaaagcgguguaccaaccaaggggugcauuuggagggagcgagaaugcuacaaaccuguucuuacuggaacugcuuggugcaggagagcuggcacuaaccaugcgaagcaagaagcuccccaucaacguuaccaccggggaagaacagcaagucagccuugaauccguggauguguacuuucaggauguuuucggcaccauguggugucaccacgcugagaugcagaaucccguguaucucauacccgagacuguacccuacaucaagugggauaauugcaauucaacuaauauuacggcuguggugagggcccagggcuuggacgugacucugcccuuaucucuaccuacuucugcccaagacuccaauuucucugucaagaccgagaugcucgggaaugagaucgauaucgagugcaucauggaggacggugagauaagccagguucugcccggcgacaacaaguucaauaucacuuguucuggcuacgagucccaugugccuagugguggcauacucacaaguacuucucccguagccacgcccauucccggaaccggauacgccuacagucugcgucugaccccacggccuguguccagauuccuggguaacaauaguaucuuauacguguuuuauagcggaaacggcccuaaagcguccggaggggacuauuguauucagaguaauaucguuuucucugaugagauuccugccagucaggacaugccgacaaacacaacugauauuaccuacgugggcgacaaugccacguauucagugcccauggucacgagcgaggacgccaauucaccaaauguuacuguaacagcuuucugggccuggccaaauaacacugagacugacuucaaauguaaguggacuuugaccucuggaacuccgucggguugcgagaauaucagcggggccuuugcuuccaacaggacuuucgacaucacugucucagggcuggggacagcaccgaagacauuaaucauaacacggaccgccaccaacgccacgacuacaacccauaaggugaucuuuuccaaggcaccugaguccaccacuaccuccccgacucuuaacacuacgggcuucgcugaucccaacaccacuacgggguugccuagcucgacacaugugccgacgaaccugacugccccugcaucgaccgggcccacaguuucgaccgccgaugugacaucaccaacgcccgcagguacaaccucaggcgccagcccagugaccccuuccccaagccccugggauaauggaacggaguccacgccuccccagaacgcaaccaguccucaggcgcccagcgggcagaagacugcggugccaacugugaccagcaccgguggcaaggccaacucaacaacuggaggcaagcauacgacggggcacggcgcccggaccuccacugaacccacgaccgauuacggaggugacagcacaacaccgcggccacgauauaaugccaccacuuaucugccaccauccacaagcuccaagcugcggccacgguggaccuucacaagcccacccgugacgacugcccaagcgacggugccagugccaccuacaagccagccacgcuucuccaaccuuaguaugcucguucuccagugggccagccuugcuguucugacccuccuccugcugcucgugauggccgauugcgccuuuaggagaaaccucagcacuagccacacguacaccacgccgcccuacgaugacgccgagacuuacguc

SEQ ID NO: 3 - RNA, 119 nt, Artificial Sequence
  Synthetic
ugauaauaggcuggagccucgguggccuagcuucuugccccuugggccuccccccagccccuccuccccuuccugcacccguacccccguggucuuugaauaaagucugagugggcggc

SEQ ID NO: 4 - protein, 658 aa, Artificial Sequence
  Synthetic
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 5 - RNA, 2118 nt, Artificial Sequence
  Synthetic
augcaguuguugugcguguucugccucguguuacucugggaggugggcgccgccagccuuagcgaggugaagcuccacuuggacaucgagggccacgccagccacuacaccauccccuggaccgagcucauggccaaggugcccggccuuagccccgaggcccuguggcgggaggccaacgugaccgaggaccuggccagcaugcugaaccgguacaagcugaucuacaagaccagcggcacccugggcaucgcccuggccgagcccguggacauccccgccguuagcgaaggcagcaugcagguggacgccagcaaggugcaccccggcgugaucagcggccugaacagccccgccuguauguugagcgccccacuggagaagcagcuguucuacuacaucggcaccaugcugcccaacacccggccccacagcuacguguucuaccagcugcggugccaccugagcuacguugcccugagcaucaacggcgacaaguuccaguacaccggcgccaugaccagcaaguuccugaugggcaccuacaagcgggucaccgagaagggcgacgagcacgugcugucacugguguucggcaagaccaaggaccugcccgaccugcggggccccuucagcuacccuaguuugaccagcgcccagagcggcgacuacagcuuggugaucgugaccaccuucgugcacuacgccaacuuccacaacuacuucgugcccaaccugaaggacauguucagccgggccgugaccaugacugccgcuucuuacgcccgguacgugcugcagaagcugguccugcuggagaugaagggcggcugccgggagcccgagcuggacaccgaaacacugaccaccauguucgaggugagcguggccuucuucaaggugggucacgcggugggcgaaaccggcaacggcugcguggacuuacgcuggcuggccaagagcuucuucgagcugaccgugcugaaggauaucaucggcaucugcuacggcgccaccgugaagggcaugcagagcuacggccuggagcggcuggccgccaugcuuauggcaacagugaagauggaggagcugggacaccugacaacagagaagcaggaguacgcccugagacuggccacagugggcuacccaaaggccggcguguacaguggacugaucggcggcgcaaccagcgugcugcuauccgcuuacaaccggcacccgcuguuccagccccugcacaccgugaugcgggaaacccuguucaucggaagccacgucgugcugcgggagcugaggcugaacguaaccacccagggcccuaaucuggcccuguaucagcuccucaguaccgcccugugcagcgcccuugagaucggcgaggugcucagaggccuggcccucgguaccgagagcggccucuucagcccaugcuacuuaagccugcgguucgaccugacccgggacaaguugcugagcauggccccgcaggaggccacacuggaccaggcagcuguauccaacgccguggacggcuuccugggcagacugucccuggaacgggaggaccgggacgccuggcaccugccugccuacaaguguguggaucggcuggacaaggugcugaugaucaucccucugauuaaugucaccuucaucaucagcagcgaccgggaggugcggggauccgcccucuacgaggccagcaccaccuaucugagcagcagccuguuccugucuccugugaucaugaacaagugcagccagggcgccguggccggcgagccccggcagauccccaagauccagaacuucacccggacccagaagucuugcaucuucugcggcuucgcccuuuuguccuacgacgagaaggagggcuuggagacuacaaccuacaucaccagccaggaggugcagaacagcauccugucaucuaauuacuucgacuucgacaaccugcacguucauuaccugcuccucaccaccaacgguaccgucauggaaaucgccggacuguacgaggagcgggcccauguugugcuggccaucauccuguacuucaucgcuuucgcacuuggcaucuuccuggugcacaagaucgugauguucuuccug

SEQ ID NO: 6 - protein, 706 aa, Artificial Sequence
  Synthetic
MQLLCVFCLVLLWEVGAASLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHVVLAIILYFIAFALGIFLVHKIVMFFL

SEQ ID NO: 7 - RNA, 411 nt, Artificial Sequence
  Synthetic
augagagccgugggcguguuccuggccaucugccuggugaccaucuucgugcugcccaccuggggcaacugggccuaccccugcugccacgugacccagcugagagcccagcaccugcuggcccuggagaacaucagcgacaucuaccuggugagcaaccagaccugcgacggcuucagccuggccagccugaacagccccaagaacggcagcaaccagcuggugaucagcagaugcgccaacggccugaacguggugagcuucuucaucagcauccugaagagaagcagcagcgcccugaccggccaccugagagagcugcugaccacccuggagacccuguacggcagcuucagcguggaggaccuguucggcgccaaccugaacagauacgccuggcacagaggcggc

SEQ ID NO: 8 - protein, 137 aa, Artificial Sequence
  Synthetic
MRAVGVFLAICLVTIFVLPTWGNWAYPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGG

SEQ ID NO: 9 - RNA, 612 nt, Artificial Sequence
  Synthetic
augacccggcggcggguguuaagcgugguggugcucuuagccgccuuagccugccggcuuggcgccaucaccugggugcccaagcccaacguggagguguggcccguggacccgccaccucccgugaacuucaacaagaccgccgagcaggaguacggcgacaaggaggugaagcugccccacuggacgccgacccugcacaccuuccaggugccccagaacuacaccaaggccaacugcaccuacugcaacaccagagaguacaccuucagcuacaagggcugcugcuucuacuucaccaagaagaagcacaccuggaacggcugcuuccaggccugcgccgagcuguaccccugcaccuacuucuacggccccaccccagacauccugcccguggugaccagaaaccugaacgccaucgagagccugugggugggcguguacagagugggcgagggcaacuggaccagccuggacggcggcaccuucaagguguaccagaucuucggcagccacugcaccuacgugagcaaguucagcaccgugcccgugagccaccacgagugcagcuuccugaagcccugccugugcgugagccagagaagcaacagc

SEQ ID NO: 10 - protein, 204 aa, Artificial Sequence
  Synthetic
MTRRRVLSVVVLLAALACRLGAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNS

SEQ ID NO: 11 - RNA, 2571 nt, Artificial Sequence
  Synthetic
augacccggcggcggguguuaagcgugguggugcucuuagccgccuuagccugccggcuuggcgcccagacuccggagcagcccgcuccucccgccaccaccguccagcccaccgccacccgccagcagaccuccuuccccuuccgcgucugcgagcucuccucccacggcgaccucuuccgcuucuccuccgacauccagugccccuccuucggcacccgcgagaaccacaccgagggccuccucauggucuucaaggacaacaucauccccuacuccuucaagguccgcuccuacaccaagaucgucaccaacauccucaucuacaacggcugguacgccgacucugugacuaaucgccacgaggagaaguucuccgucgacuccuacgagacugaccagauggacaccaucuaccagugcuacaacgccgucaagaugaccaaggacggccucacccgcgucuacgucgaccgcgacggcgucaacaucaccgucaaccucaagccuacaggcggccucgccaacggcguccgccgcuacgccucccagaccgagcucuacgacgccccuggcuggcucaucuggaccuaccgcaccaggaccacugucaauugccucaucaccgacaugauggccaaguccaacuccccuuucgacuucuucgugaccaccacaggccagaccgucgagaugucgccauucuaugacggcaagaacaaggagacuuuccacgagagagcggacucuuuccauguccgcaccaacuacaagauugucgacuacgacaaccgcggcaccaauccacagggcgagcgccgcgccuuccucgacaagggcaccuacacccucuccuggaagcucgagaaccgcaccgccuacuguccucuccagcacuggcagaccuucgauuccaccaucgccaccgagacuggcaaguccauccauuucgucaccgacgaaggaacaucuucauucgucacaaauacaaccgugggcaucgagcuccccgacgccuucaagugcaucgaggagcaggucaacaagaccaugcacgagaaguacgaggccguccaggaccgguauacaaagggccaggaggccaucaccuacuucaucaccagcggaggucugcuucucgccuggcuuccacucacaccucgcucccuggccacagugaagaaccucacugaguugaccaccccaacaaguuccccaccuaguaguccuagcccuccugcuccuuccgccgcccgcggcuccacgccugccgccguccugcggagacggagaagggacgccggcaacgcuaccacuccugugccgccgacagccccuggaaagucccuagguacccucaacaacccagcgacaguucagauccaguucgccuacgacucucucaggcgccagaucaaccgcaugcuuggugaccuugcccgcgccuggugccucgagcagaagcgccagaacauggugcuucgugaacuaacaaagauuaauccuacuacagugauguccuccaucuacggcaaggccgucgccgccaagcgccucggcgacgucaucuccgucucccagugcgucccagugaaccaggcuaccgugacccucaggaaguccaugagagugccaggcuccgagacaaugugcuacucccgcccacucgucuccuucuccuucaucaacgacacaaagaccuacgagggccaguuaggcacugacaacgagaucuucuugacuaagaagaugacugagguaugccaggcaacuucucaauacuacuuccagucuggaaaugagauccacguauauaacgacuaccaccacuucaagacuauugaacucgacggaauugccacccuccaaacauucaucucacugaauaccucccucaucgagaacaucgacuucgccucccucgagcuguauagcagagacgagcagcgcgccuccaacgucuucgaccucgagggcaucuuccgcgaguacaacuuccaggcacagaacauagccggccuccguaaggaucuggacaaugccguguccaacggccgcaaccaguucgucgacgguuugggugaacucauggacucucugggcuccgucggccaguccauaacuaacuuagucucuacggugggaggccuauucagcagccuggugagcggcuucaucucuuucuucaagaaccccuucggcggcaugcucauccucguccucgucgccggcgucgucauacuggugaucucacucacaaggaggacgcgccaaaugucccagcagccagugcagaugcucuacccaggcauagacgagcucgcucagcagcacgcgucgggugagggaccaggcaucaauccuaucuccaagacugagcugcaagccauuaugcucgcccuccacgagcagaaccaggaacagaagcgggccgcccagcgagcugccggccccuccgucgccaguagggcacuacaagccgcccgggaccgcuucccaggccugagaaggaggagauaccacgacccagagacagccgcugcccuccuuggcgaagcagaaaccgaguuc

SEQ ID NO: 12 - protein, 857 aa, Artificial Sequence
  Synthetic
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 13 - RNA, 669 nt, Artificial Sequence
  Synthetic
auggugagcuucaagcaggugagagugccccuguucaccgccaucgcccuggugaucgugcugcugcuggccuacuuccugccacccagagugagaggcggcggcagaguggccgccgccgccaucaccugggugcccaagcccaacguggagguguggcccguggacccgccaccucccgugaacuucaacaagaccgccgagcaggaguacggcgacaaggaggugaagcugccccacuggacgccgacccugcacaccuuccaggugccccagaacuacaccaaggccaacugcaccuacugcaacaccagagaguacaccuucagcuacaagggcugcugcuucuacuucaccaagaagaagcacaccuggaacggcugcuuccaggccugcgccgagcuguaccccugcaccuacuucuacggccccaccccagacauccugcccguggugaccagaaaccugaacgccaucgagagccugugggugggcguguacagagugggcgagggcaacuggaccagccuggacggcggcaccuucaagguguaccagaucuucggcagccacugcaccuacgugagcaaguucagcaccgugcccgugagccaccacgagugcagcuuccugaagcccugccugugcgugagccagagaagcaacagc

SEQ ID NO: 14 - protein, 223 aa, Artificial Sequence
  Synthetic
MVSFKQVRVPLFTAIALVIVLLLAYFLPPRVRGGGRVAAAAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNS

SEQ ID NO: 15 - RNA, 2595 nt, Artificial Sequence
  Synthetic
auggaggcagcacugcuggucugccaguauacaauccagucgcucauucaucugacaggcgaggaccccggcuucuuuaacguggaaaucccagaguucccuuucuauccaaccuguaacguuugcacugcugacgugaacgugacaaucaauuucgacgugggaggaaagaagcaucagcuugaucuggauuucggccaacugacgccacacaccaaagcgguguaccaaccaaggggugcauuuggagggagcgagaaugcuacaaaucuguucuuacuggaacugcuuggugcaggagagcuggcacuaaccaugcgaagcaagaagcuccccaucaacguuaccaccggggaagaacagcaagucagccuugaauccguggauguguacuuucaggauguuuucggcaccauguggugucaccacgcugagaugcagaaucccguguaucucauacccgagacuguacccuacaucaagugggauaauugcaauucaacuaauauuacggcuguggugagggcccagggcuuggacgugacucugcccuuaucucuaccuacuucugcccaagacuccaauuucucugucaagaccgagaugcucgggaaugagaucgauaucgagugcaucauggaggacggugagauaagccagguucugcccggcgacaacaaguucaauaucacuuguucuggcuacgagucccaugugccuagugguggcauacucacaaguacuucucccguagccacgcccauucccggaaccggauacgccuacagucugcgucugaccccacggccuguguccagauuccuggguaacaauaguaucuuauacguguuuuauagcggaaacggcccuaaagcguccggaggggacuauuguauucagaguaauaucguuuucucugaugagauuccugccagucaggacaugccgacaaacacaacugauauuaccuacgugggcgacaaugccacguauucagugcccauggucacgagcgaggacgccaauucaccaaauguuacuguaacagcuuucugggccuggccaaauaacacugagacugacuucaaauguaaguggacuuugaccucuggaacuccgucggguugcgagaauaucagcggggccuuugcuuccaacaggacuuucgacaucacugucucagggcuggggacagcaccgaagacauuaaucauaacacggaccgccaccaacgccacgacuacaacccauaaggugaucuuuuccaaggcaccugaguccaccacuaccuccccgacucuuaacacuacgggcuucgcugaucccaacaccacuacgggguugccuagcucgacacaugugccgacgaaccugacugccccugcaucgaccgggcccacaguuucgaccgccgaugugacaucaccaacgcccgcagguacaaccucaggcgccagcccagugaccccuuccccaagccccugggauaauggaacggaguccaaggcuccugauaugacuuccucuaccagccccgugacuacacccacucccaacgcaacuagcccaaccccagcugugacgacgcccaccccgaacgcgacaucucccacaccugcugugacaaccccaaccccuaacgccacuagcccuacccuagguaagaccaguccgacuagcgccguuacaaccccuaccccuaacgcaaccggcccgaccgugggcgagacuuccccgcaagccaaugcgacaaaucacacauugggcgggaccucuccuacaccagucguuacaucucagccuaagaacgcuaccuccgcugucacuaccggacagcacaacaucaccagcucaagcacgaguuccaugagcuugcggccgagcucaaaucccgagacccuaucaccauccacaucagacaacaguacuucacacaugccacucuugacgagcgcucaccccaccggcggcgagaacaucacccaggugacuccggcgucuauuuccacccaccacgucagcacgucgucucccgcaccgagaccagggacgacuucucaggccagugggccuggcaacuccucuacaagcacaaagccaggcgaaguuaacgugacaaagggaacgccuccccagaacgcaaccaguccucaggcgcccagcgggcagaagacugcggugccaacugugaccagcaccgguggcaaggccaacucaacaacuggaggcaagcauacgacggggcacggcgcccggaccuccacugaacccacgaccgauuacggaggugacagcacaacaccgcggccacgauauaaugccaccacuuaucugccaccauccacaagcuccaagcugcggccacgguggaccuucacaagcccacccgugacgacugcccaagcgacggugccagugccaccuacaagccagccacgcuucuccaaccuuaguaugcucguucuccagugggccagccuugcuguucugacccuccuccugcugcucgugauggccgauugcgccuuuaggagaaaccucagcacuagccacacguacaccacgccgcccuacgaugacgccgagacuuacguc

SEQ ID NO: 16 - protein, 865 aa, Artificial Sequence
  Synthetic
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 17 - RNA, 2150 nt, Artificial Sequence
  Synthetic
gggaaauaagagagaaaagaagaguaagaagaaauauaagaccccggcgccgccaccauggaggcagcacugcuggucugccaguauacaauccagucgcucauucaucugacaggcgaggaccccggcuucuuuaacguggaaaucccagaguucccuuucuauccaaccuguaacguuugcacugcugacgugaacgugacaaucaauuucgacgugggaggaaagaagcaucagcuugaucuggauuucggccaacugacgccacacaccaaagcgguguaccaaccaaggggugcauuuggagggagcgagaaugcuacaaaccuguucuuacuggaacugcuuggugcaggagagcuggcacuaaccaugcgaagcaagaagcuccccaucaacguuaccaccggggaagaacagcaagucagccuugaauccguggauguguacuuucaggauguuuucggcaccauguggugucaccacgcugagaugcagaaucccguguaucucauacccgagacuguacccuacaucaagugggauaauugcaauucaacuaauauuacggcuguggugagggcccagggcuuggacgugacucugcccuuaucucuaccuacuucugcccaagacuccaauuucucugucaagaccgagaugcucgggaaugagaucgauaucgagugcaucauggaggacggugagauaagccagguucugcccggcgacaacaaguucaauaucacuuguucuggcuacgagucccaugugccuagugguggcauacucacaaguacuucucccguagccacgcccauucccggaaccggauacgccuacagucugcgucugaccccacggccuguguccagauuccuggguaacaauaguaucuuauacguguuuuauagcggaaacggcccuaaagcguccggaggggacuauuguauucagaguaauaucguuuucucugaugagauuccugccagucaggacaugccgacaaacacaacugauauuaccuacgugggcgacaaugccacguauucagugcccauggucacgagcgaggacgccaauucaccaaauguuacuguaacagcuuucugggccuggccaaauaacacugagacugacuucaaauguaaguggacuuugaccucuggaacuccgucggguugcgagaauaucagcggggccuuugcuuccaacaggacuuucgacaucacugucucagggcuggggacagcaccgaagacauuaaucauaacacggaccgccaccaacgccacgacuacaacccauaaggugaucuuuuccaaggcaccugaguccaccacuaccuccccgacucuuaacacuacgggcuucgcugaucccaacaccacuacgggguugccuagcucgacacaugugccgacgaaccugacugccccugcaucgaccgggcccacaguuucgaccgccgaugugacaucaccaacgcccgcagguacaaccucaggcgccagcccagugaccccuuccccaagccccugggauaauggaacggaguccacgccuccccagaacgcaaccaguccucaggcgcccagcgggcagaagacugcggugccaacugugaccagcaccgguggcaaggccaacucaacaacuggaggcaagcauacgacggggcacggcgcccggaccuccacugaacccacgaccgauuacggaggugacagcacaacaccgcggccacgauauaaugccaccacuuaucugccaccauccacaagcuccaagcugcggccacgguggaccuucacaagcccacccgugacgacugcccaagcgacggugccagugccaccuacaagccagccacgcuucuccaaccuuaguaugcucguucuccagugggccagccuugcuguucugacccuccuccugcugcucgugauggccgauugcgccuuuaggagaaaccucagcacuagccacacguacaccacgccgcccuacgaugacgccgagacuuacgucugauaauaggcuggagccucgguggccuagcuucuugccccuugggccuccccccagccccuccuccccuuccugcacccguacccccguggucuuugaauaaagucugagugggcggc

SEQ ID NO: 18 - RNA, 2294 nt, Artificial Sequence
  Synthetic
gggaaauaagagagaaaagaagaguaagaagaaauauaagaccccggcgccgccaccaugcaguuguugugcguguucugccucguguuacucugggaggugggcgccgccagccuuagcgaggugaagcuccacuuggacaucgagggccacgccagccacuacaccauccccuggaccgagcucauggccaaggugcccggccuuagccccgaggcccuguggcgggaggccaacgugaccgaggaccuggccagcaugcugaaccgguacaagcugaucuacaagaccagcggcacccugggcaucgcccuggccgagcccguggacauccccgccguuagcgaaggcagcaugcagguggacgccagcaaggugcaccccggcgugaucagcggccugaacagccccgccuguauguugagcgccccacuggagaagcagcuguucuacuacaucggcaccaugcugcccaacacccggccccacagcuacguguucuaccagcugcggugccaccugagcuacguugcccugagcaucaacggcgacaaguuccaguacaccggcgccaugaccagcaaguuccugaugggcaccuacaagcgggucaccgagaagggcgacgagcacgugcugucacugguguucggcaagaccaaggaccugcccgaccugcggggccccuucagcuacccuaguuugaccagcgcccagagcggcgacuacagcuuggugaucgugaccaccuucgugcacuacgccaacuuccacaacuacuucgugcccaaccugaaggacauguucagccgggccgugaccaugacugccgcuucuuacgcccgguacgugcugcagaagcugguccugcuggagaugaagggcggcugccgggagcccgagcuggacaccgaaacacugaccaccauguucgaggugagcguggccuucuucaaggugggucacgcggugggcgaaaccggcaacggcugcguggacuuacgcuggcuggccaagagcuucuucgagcugaccgugcugaaggauaucaucggcaucugcuacggcgccaccgugaagggcaugcagagcuacggccuggagcggcuggccgccaugcuuauggcaacagugaagauggaggagcugggacaccugacaacagagaagcaggaguacgcccugagacuggccacagugggcuacccaaaggccggcguguacaguggacugaucggcggcgcaaccagcgugcugcuauccgcuuacaaccggcacccgcuguuccagccccugcacaccgugaugcgggaaacccuguucaucggaagccacgucgugcugcgggagcugaggcugaacguaaccacccagggcccuaaucuggcccuguaucagcuccucaguaccgcccugugcagcgcccuugagaucggcgaggugcucagaggccuggcccucgguaccgagagcggccucuucagcccaugcuacuuaagccugcgguucgaccugacccgggacaaguugcugagcauggccccgcaggaggccacacuggaccaggcagcuguauccaacgccguggacggcuuccugggcagacugucccuggaacgggaggaccgggacgccuggcaccugccugccuacaaguguguggaucggcuggacaaggugcugaugaucaucccucugauuaaugucaccuucaucaucagcagcgaccgggaggugcggggauccgcccucuacgaggccagcaccaccuaucugagcagcagccuguuccugucuccugugaucaugaacaagugcagccagggcgccguggccggcgagccccggcagauccccaagauccagaacuucacccggacccagaagucuugcaucuucugcggcuucgcccuuuuguccuacgacgagaaggagggcuuggagacuacaaccuacaucaccagccaggaggugcagaacagcauccugucaucuaauuacuucgacuucgacaaccugcacguucauuaccugcuccucaccaccaacgguaccgucauggaaaucgccggacuguacgaggagcgggcccauguugugcuggccaucauccuguacuucaucgcuuucgcacuuggcaucuuccuggugcacaagaucgugauguucuuccugugauaauaggcuggagccucgguggccuagcuucuugccccuugggccuccccccagccccuccuccccuuccugcacccguacccccguggucuuugaauaaagucugagugggcggc

SEQ ID NO: 19 - RNA, 587 nt, Artificial Sequence
  Synthetic
gggaaauaagagagaaaagaagaguaagaagaaauauaagaccccggcgccgccaccaugagagccgugggcguguuccuggccaucugccuggugaccaucuucgugcugcccaccuggggcaacugggccuaccccugcugccacgugacccagcugagagcccagcaccugcuggcccuggagaacaucagcgacaucuaccuggugagcaaccagaccugcgacggcuucagccuggccagccugaacagccccaagaacggcagcaaccagcuggugaucagcagaugcgccaacggccugaacguggugagcuucuucaucagcauccugaagagaagcagcagcgcccugaccggccaccugagagagcugcugaccacccuggagacccuguacggcagcuucagcguggaggaccuguucggcgccaaccugaacagauacgccuggcacagaggcggcugauaauaggcuggagccucgguggccuagcuucuugccccuugggccuccccccagccccuccuccccuuccugcacccguacccccguggucuuugaauaaagucugagugggcggc

SEQ ID NO: 20 - RNA, 788 nt, Artificial Sequence
  Synthetic
gggaaauaagagagaaaagaagaguaagaagaaauauaagaccccggcgccgccaccaugacccggcggcggguguuaagcgugguggugcucuuagccgccuuagccugccggcuuggcgccaucaccugggugcccaagcccaacguggagguguggcccguggacccgccaccucccgugaacuucaacaagaccgccgagcaggaguacggcgacaaggaggugaagcugccccacuggacgccgacccugcacaccuuccaggugccccagaacuacaccaaggccaacugcaccuacugcaacaccagagaguacaccuucagcuacaagggcugcugcuucuacuucaccaagaagaagcacaccuggaacggcugcuuccaggccugcgccgagcuguaccccugcaccuacuucuacggccccaccccagacauccugcccguggugaccagaaaccugaacgccaucgagagccugugggugggcguguacagagugggcgagggcaacuggaccagccuggacggcggcaccuucaagguguaccagaucuucggcagccacugcaccuacgugagcaaguucagcaccgugcccgugagccaccacgagugcagcuuccugaagcccugccugugcgugagccagagaagcaacagcugauaauaggcuggagccucgguggccuagcuucuugccccuugggccuccccccagccccuccuccccuuccugcacccguacccccguggucuuugaauaaagucugagugggcggc

SEQ ID NO: 21 - RNA, 2747 nt, Artificial Sequence
  Synthetic
gggaaauaagagagaaaagaagaguaagaagaaauauaagaccccggcgccgccaccaugacccggcggcggguguuaagcgugguggugcucuuagccgccuuagccugccggcuuggcgcccagacuccggagcagcccgcuccucccgccaccaccguccagcccaccgccacccgccagcagaccuccuuccccuuccgcgucugcgagcucuccucccacggcgaccucuuccgcuucuccuccgacauccagugccccuccuucggcacccgcgagaaccacaccgagggccuccucauggucuucaaggacaacaucauccccuacuccuucaagguccgcuccuacaccaagaucgucaccaacauccucaucuacaacggcugguacgccgacucugugacuaaucgccacgaggagaaguucuccgucgacuccuacgagacugaccagauggacaccaucuaccagugcuacaacgccgucaagaugaccaaggacggccucacccgcgucuacgucgaccgcgacggcgucaacaucaccgucaaccucaagccuacaggcggccucgccaacggcguccgccgcuacgccucccagaccgagcucuacgacgccccuggcuggcucaucuggaccuaccgcaccaggaccacugucaauugccucaucaccgacaugauggccaaguccaacuccccuuucgacuucuucgugaccaccacaggccagaccgucgagaugucgccauucuaugacggcaagaacaaggagacuuuccacgagagagcggacucuuuccauguccgcaccaacuacaagauugucgacuacgacaaccgcggcaccaauccacagggcgagcgccgcgccuuccucgacaagggcaccuacacccucuccuggaagcucgagaaccgcaccgccuacuguccucuccagcacuggcagaccuucgauuccaccaucgccaccgagacuggcaaguccauccauuucgucaccgacgaaggaacaucuucauucgucacaaauacaaccgugggcaucgagcuccccgacgccuucaagugcaucgaggagcaggucaacaagaccaugcacgagaaguacgaggccguccaggaccgguauacaaagggccaggaggccaucaccuacuucaucaccagcggaggucugcuucucgccuggcuuccacucacaccucgcucccuggccacagugaagaaccucacugaguugaccaccccaacaaguuccccaccuaguaguccuagcccuccugcuccuuccgccgcccgcggcuccacgccugccgccguccugcggagacggagaagggacgccggcaacgcuaccacuccugugccgccgacagccccuggaaagucccuagguacccucaacaacccagcgacaguucagauccaguucgccuacgacucucucaggcgccagaucaaccgcaugcuuggugaccuugcccgcgccuggugccucgagcagaagcgccagaacauggugcuucgugaacuaacaaagauuaauccuacuacagugauguccuccaucuacggcaaggccgucgccgccaagcgccucggcgacgucaucuccgucucccagugcgucccagugaaccaggcuaccgugacccucaggaaguccaugagagugccaggcuccgagacaaugugcuacucccgcccacucgucuccuucuccuucaucaacgacacaaagaccuacgagggccaguuaggcacugacaacgagaucuucuugacuaagaagaugacugagguaugccaggcaacuucucaauacuacuuccagucuggaaaugagauccacguauauaacgacuaccaccacuucaagacuauugaacucgacggaauugccacccuccaaacauucaucucacugaauaccucccucaucgagaacaucgacuucgccucccucgagcuguauagcagagacgagcagcgcgccuccaacgucuucgaccucgagggcaucuuccgcgaguacaacuuccaggcacagaacauagccggccuccguaaggaucuggacaaugccguguccaacggccgcaaccaguucgucgacgguuugggugaacucauggacucucugggcuccgucggccaguccauaacuaacuuagucucuacggugggaggccuauucagcagccuggugagcggcuucaucucuuucuucaagaaccccuucggcggcaugcucauccucguccucgucgccggcgucgucauacuggugaucucacucacaaggaggacgcgccaaaugucccagcagccagugcagaugcucuacccaggcauagacgagcucgcucagcagcacgcgucgggugagggaccaggcaucaauccuaucuccaagacugagcugcaagccauuaugcucgcccuccacgagcagaaccaggaacagaagcgggccgcccagcgagcugccggccccuccgucgccaguagggcacuacaagccgcccgggaccgcuucccaggccugagaaggaggagauaccacgacccagagacagccgcugcccuccuuggcgaagcagaaaccgaguucugauaauaggcuggagccucgguggccuagcuucuugccccuugggccuccccccagccccuccuccccuuccugcacccguacccccguggucuuugaauaaagucugagugggcggc

SEQ ID NO: 22 - RNA, 845 nt, Artificial Sequence
  Synthetic
gggaaauaagagagaaaagaagaguaagaagaaauauaagaccccggcgccgccaccauggugagcuucaagcaggugagagugccccuguucaccgccaucgcccuggugaucgugcugcugcuggccuacuuccugccacccagagugagaggcggcggcagaguggccgccgccgccaucaccugggugcccaagcccaacguggagguguggcccguggacccgccaccucccgugaacuucaacaagaccgccgagcaggaguacggcgacaaggaggugaagcugccccacuggacgccgacccugcacaccuuccaggugccccagaacuacaccaaggccaacugcaccuacugcaacaccagagaguacaccuucagcuacaagggcugcugcuucuacuucaccaagaagaagcacaccuggaacggcugcuuccaggccugcgccgagcuguaccccugcaccuacuucuacggccccaccccagacauccugcccguggugaccagaaaccugaacgccaucgagagccugugggugggcguguacagagugggcgagggcaacuggaccagccuggacggcggcaccuucaagguguaccagaucuucggcagccacugcaccuacgugagcaaguucagcaccgugcccgugagccaccacgagugcagcuuccugaagcccugccugugcgugagccagagaagcaacagcugauaauaggcuggagccucgguggccuagcuucuugccccuugggccuccccccagccccuccuccccuuccugcacccguacccccguggucuuugaauaaagucugagugggcggc

SEQ ID NO: 23 - RNA, 2771 nt, Artificial Sequence
  Synthetic
gggaaauaagagagaaaagaagaguaagaagaaauauaagaccccggcgccgccaccauggaggcagcacugcuggucugccaguauacaauccagucgcucauucaucugacaggcgaggaccccggcuucuuuaacguggaaaucccagaguucccuuucuauccaaccuguaacguuugcacugcugacgugaacgugacaaucaauuucgacgugggaggaaagaagcaucagcuugaucuggauuucggccaacugacgccacacaccaaagcgguguaccaaccaaggggugcauuuggagggagcgagaaugcuacaaaucuguucuuacuggaacugcuuggugcaggagagcuggcacuaaccaugcgaagcaagaagcuccccaucaacguuaccaccggggaagaacagcaagucagccuugaauccguggauguguacuuucaggauguuuucggcaccauguggugucaccacgcugagaugcagaaucccguguaucucauacccgagacuguacccuacaucaagugggauaauugcaauucaacuaauauuacggcuguggugagggcccagggcuuggacgugacucugcccuuaucucuaccuacuucugcccaagacuccaauuucucugucaagaccgagaugcucgggaaugagaucgauaucgagugcaucauggaggacggugagauaagccagguucugcccggcgacaacaaguucaauaucacuuguucuggcuacgagucccaugugccuagugguggcauacucacaaguacuucucccguagccacgcccauucccggaaccggauacgccuacagucugcgucugaccccacggccuguguccagauuccuggguaacaauaguaucuuauacguguuuuauagcggaaacggcccuaaagcguccggaggggacuauuguauucagaguaauaucguuuucucugaugagauuccugccagucaggacaugccgacaaacacaacugauauuaccuacgugggcgacaaugccacguauucagugcccauggucacgagcgaggacgccaauucaccaaauguuacuguaacagcuuucugggccuggccaaauaacacugagacugacuucaaauguaaguggacuuugaccucuggaacuccgucggguugcgagaauaucagcggggccuuugcuuccaacaggacuuucgacaucacugucucagggcuggggacagcaccgaagacauuaaucauaacacggaccgccaccaacgccacgacuacaacccauaaggugaucuuuuccaaggcaccugaguccaccacuaccuccccgacucuuaacacuacgggcuucgcugaucccaacaccacuacgggguugccuagcucgacacaugugccgacgaaccugacugccccugcaucgaccgggcccacaguuucgaccgccgaugugacaucaccaacgcccgcagguacaaccucaggcgccagcccagugaccccuuccccaagccccugggauaauggaacggaguccaaggcuccugauaugacuuccucuaccagccccgugacuacacccacucccaacgcaacuagcccaaccccagcugugacgacgcccaccccgaacgcgacaucucccacaccugcugugacaaccccaaccccuaacgccacuagcccuacccuagguaagaccaguccgacuagcgccguuacaaccccuaccccuaacgcaaccggcccgaccgugggcgagacuuccccgcaagccaaugcgacaaaucacacauugggcgggaccucuccuacaccagucguuacaucucagccuaagaacgcuaccuccgcugucacuaccggacagcacaacaucaccagcucaagcacgaguuccaugagcuugcggccgagcucaaaucccgagacccuaucaccauccacaucagacaacaguacuucacacaugccacucuugacgagcgcucaccccaccggcggcgagaacaucacccaggugacuccggcgucuauuuccacccaccacgucagcacgucgucucccgcaccgagaccagggacgacuucucaggccagugggccuggcaacuccucuacaagcacaaagccaggcgaaguuaacgugacaaagggaacgccuccccagaacgcaaccaguccucaggcgcccagcgggcagaagacugcggugccaacugugaccagcaccgguggcaaggccaacucaacaacuggaggcaagcauacgacggggcacggcgcccggaccuccacugaacccacgaccgauuacggaggugacagcacaacaccgcggccacgauauaaugccaccacuuaucugccaccauccacaagcuccaagcugcggccacgguggaccuucacaagcccacccgugacgacugcccaagcgacggugccagugccaccuacaagccagccacgcuucuccaaccuuaguaugcucguucuccagugggccagccuugcuguucugacccuccuccugcugcucgugauggccgauugcgccuuuaggagaaaccucagcacuagccacacguacaccacgccgcccuacgaugacgccgagacuuacgucugauaauaggcuggagccucgguggccuagcuucuugccccuugggccuccccccagccccuccuccccuuccugcacccguacccccguggucuuugaauaaagucugagugggcggc

SEQ ID NO: 24 - RNA, 47 nt, Artificial Sequence
  Synthetic
gggaaauaagagagaaaagaagaguaagaagaaauauaagagccacc

SEQ ID NO: 25 - RNA, 119 nt, Artificial Sequence
  Synthetic
ugauaauaggcuggagccucgguggccaugcuucuugccccuugggccuccccccagccccuccuccccuuccugcacccguacccccguggucuuugaauaaagucugagugggcggc

SEQ ID NO: 26 - protein, 30 aa, Artificial Sequence
  Synthetic
MDSKGSSQKGSRLLLLLVVSNLLLPQGVVG

SEQ ID NO: 27 - protein, 18 aa, Artificial Sequence
  Synthetic
MDWTWILFLVAAATRVHS

SEQ ID NO: 28 - protein, 20 aa, Artificial Sequence
  Synthetic
METPAQLLFLLLLWLPDTTG

SEQ ID NO: 29 - protein, 24 aa, Artificial Sequence
  Synthetic
MLGSNSGQRVVFTILLLLVAPAYS

SEQ ID NO: 30 - protein, 17 aa, Artificial Sequence
  Synthetic
MKCLLYLAFLFIGVNCA

SEQ ID NO: 31 - protein, 15 aa, Artificial Sequence
  Synthetic
MWLVSLAIVTACAGA

SEQ ID NO: 32 - RNA, 9 nt, Artificial Sequence
  Synthetic
uuauuuaww

SEQ ID NO: 33 - RNA, 9 nt, Artificial Sequence
  Synthetic
ccrccaugg

SEQ ID NO: 34 - RNA, 11 nt, Artificial Sequence
  Synthetic
gggauccuacc